SEQUENCE LISTING (39 sequences)

SEQ ID NO: 1 - DNA, 3218 bp, Saccharomyces cerevisiae
aaataggaatgtgataccttctattgcatgcaaagatagtgtaggaggcgctgctattgccaaagacttttgagaccgcttgctgtttcattatagttgaggagttctcgaagacgagaaattagcagttttcggtgtttagtaatcgcgctagcatgctaggacaatttaactgcaaaattttgatacgatagtgatagtaaatggaaggtaaaaataacatagacctatcaataagcaatgtctctcagaataaaagcacttgatgcatcagtggttaacaaaattgctgcaggtgagatcataatatcccccgtaaatgctctcaaagaaatgatggagaattccatcgatgcgaatgctacaatgattgatattctagtcaaggaaggaggaattaaggtacttcaaataacagataacggatctggaattaataaagcagacctgccaatcttatgtgagcgattcacgacgtccaaattacaaaaattcgaagatttgagtcagattcaaacgtatggattccgaggagaagctttagccagtatctcacatgtggcaagagtcacagtaacgacaaaagttaaagaagacagatgtgcatggagagtttcatatgcagaaggtaagatgttggaaagccccaaacctgttgctggaaaagacggtaccacgatcctagttgaagacctttttttcaatattccttctagattaagggccttgaggtcccataatgatgaatactctaaaatattagatgttgtcgggcgatacgccattcattccaaggacattggcttttcttgtaaaaagttcggagactctaattattctttatcagttaaaccttcatatacagtccaggataggattaggactgtgttcaataaatctgtggcttcgaatttaattacttttcatatcagcaaagtagaagatttaaacctggaaagcgttgatggaaaggtgtgtaatttgaatttcatatccaaaaagtccatttcattaatttttttcattaataatagactagtgacatgtgatcttctaagaagagctttgaacagcgtttactccaattatctgccaaagggcttcagaccttttatttatttgggaattgttatagatccggcggctgttgatgttaacgttcacccgacaaagagagaggttcgtttcctgagccaagatgagatcatagagaaaatcgccaatcaattgcacgccgaattatctgccattgatacttcacgtactttcaaggcttcttcaatttcaacaaacaagccagagtcattgataccatttaatgacaccatagaaagtgataggaataggaagagtctccgacaagcccaagtggtagagaattcatatacgacagccaatagtcaactaaggaaagcgaaaagacaagagaataaactagtcagaatagatgcttcacaagctaaaattacgtcatttttatcctcaagtcaacagttcaactttgaaggatcgtctacaaagcgacaactgagtgaacccaaggtaacaaatgtaagccactcccaagaggcagaaaagctgacactaaatgaaagcgaacaaccgcgtgatgccaatacaatcaatgataatgacttgaaggatcaacctaagaagaaacaaaagttgggggattataaagttccaagcattgccgatgacgaaaagaatgcactcccgatttcaaaagacgggtatattagagtacctaaggagcgagttaatgttaatcttacgagtatcaagaaattgcgtgaaaaagtagatgattcgatacatcgagaactaacagacatttttgcaaatttgaattacgttggggttgtagatgaggaaagaagattagccgctattcagcatgacttaaagctttttttaatagattacggatctgtgtgctatgagctattctatcagattggtttgacagacttcgcaaactttggtaagataaacctacagagtacaaatgtgtcagatgatatagttttgtataatctcctatcagaatttgacgagttaaatgacgatgcttccaaagaaaaaataattagtaaaatatgggacatgagcagtatgctaaatgagtactattccatagaattggtgaatgatggtctagataatgacttaaagtctgtgaagctaaaatctctaccactacttttaaaaggctacattccatctctggtcaagttaccattttttatatatcgcctgggtaaagaagttgattgggaggatgaacaagagtgtctagatggtattttaagagagattgcattactctatatacctgatatggttccgaaagtcgatacactcgatgcatcgttgtcagaagacgaaaaagcccagtttataaatagaaaggaacacatatcctcattactagaacacgttctcttcccttgtatcaaacgaaggttcctggcccctagacacattctcaaggatgtcgtggaaatagccaaccttccagatctatacaaagtttttgagaggtgttaactttaaaacgttttggctgtaataccaaagtttttgtttatttcctgagtgtgattgtgtttcatttgaaagtgtatgccctttcctttaacgattcatccgcgagatttcaaaggatatgaaatatggttgcagttaggaaagtatgtcagaaatgtatattcggattgaaactcttctaatagttctgaagtcacttggttccgtattgttttcgtcctcttcctcaagcaacgattcttgtctaagcttattcaacggtaccaaagacccgagtccttttatgagagaaaacatttcatcatttttcaactcaattatcttaatatcattttgtagtattttgaaaacaggatggtaaaacgaatcacctgaatctagaagctgtaccttgtcccataaaagttttaatttactgagcctttcggtcaagtaaactagtttatctagttttgaaccgaatattgtgggcagatttgcagtaagttcagttagatctactaaaagttgtttgacagcagccgattccacaaaaatttggtaaaaggagatgaaagagacctcgcgcgtaatggtttgcatcaccatcggatgtctgttgaaaaactcactttttgcatggaagttattaacaataagactaatgattaccttagaataatgtataa

SEQ ID NO: 2 - protein, 769 aa, Saccharomyces cerevisiae
MSLRIKALDASVVNKIAAGEIIISPVNALKEMMENSIDANATMIDILVKEGGIKVLQITDNGSGINKADLPILCERFTTSKLQKFEDLSQIQTYGFRGEALASISHVARVTVTTKVKEDRCAWRVSYAEGKMLESPKPVAGKDGTTILVEDLFFNIPSRLRALRSHNDEYSKILDVVGRYAIHSKDIGFSCKKFGDSNYSLSVKPSYTVQDRIRTVFNKSVASNLITFHISKVEDLNLESVDGKVCNLNFISKKSISLIFFINNRLVTCDLLRRALNSVYSNYLPKGFRPFIYLGIVIDPAAVDVNVHPTKREVRFLSQDEIIEKIANQLHAELSAIDTSRTFKASSISTNKPESLIPFNDTIESDRNRKSLRQAQVVENSYTTANSQLRKAKRQENKLVRIDASQAKITSFLSSSQQFNFEGSSTKRQLSEPKVTNVSHSQEAEKLTLNESEQPRDANTINDNDLKDQPKKKQKLGDYKVPSIADDEKNALPISKDGYIRVPKERVNVNLTSIKKLREKVDDSIHRELTDIFANLNYVGVVDEERRLAAIQHDLKLFLIDYGSVCYELFYQIGLTDFANFGKINLQSTNVSDDIVLYNLLSEFDELNDDASKEKIISKIWDMSSMLNEYYSIELVNDGLDNDLKSVKLKSLPLLLKGYIPSLVKLPFFIYRLGKEVDWEDEQECLDGILREIALLYIPDMVPKVDTLDASLSEDEKAQFINRKEHISSLLEHVLFPCIKRRFLAPRHILKDVVEIANLPDLYKVFERC

SEQ ID NO: 3 - DNA, 3056 bp, Mus musculus
gaattccggtgaaggtcctgaagaatttccagattcctgagtatcattggaggagacagataacctgtcgtcaggtaacgatggtgtatatgcaacagaaatgggtgttcctggagacgcgtcttttcccgagagcggcaccgcaactctcccgcggtgactgtgactggaggagtcctgcatccatggagcaaaccgaaggcgtgagtacagaatgtgctaaggccatcaagcctattgatgggaagtcagtccatcaaatttgttctgggcaggtgatactcagtttaagcaccgctgtgaaggagttgatagaaaatagtgtagatgctggtgctactactattgatctaaggcttaaagactatggggtggacctcattgaagtttcagacaatggatgtggggtagaagaagaaaactttgaaggtctagctctgaaacatcacacatctaagattcaagagtttgccgacctcacgcaggttgaaactttcggctttcggggggaagctctgagctctctgtgtgcactaagtgatgtcactatatctacctgccacgggtctgcaagcgttgggactcgactggtgtttgaccataatgggaaaatcacccagaaaactccctacccccgacctaaaggaaccacagtcagtgtgcagcacttattttatacactacccgtgcgttacaaagagtttcagaggaacattaaaaaggagtattccaaaatggtgcaggtcttacaggcgtactgtatcatctcagcaggcgtccgtgtaagctgcactaatcagctcggacaggggaagcggcacgctgtggtgtgcacaagcggcacgtctggcatgaaggaaaatatcgggtctgtgtttggccagaagcagttgcaaagcctcattccttttgttcagctgccccctagtgacgctgtgtgtgaagagtacggcctgagcacttcaggacgccacaaaaccttttctacgtttcgggcttcatttcacagtgcacgcacggcgccgggaggagtgcaacagacaggcagtttttcttcatcaatcagaggccctgtgacccagcaaaggtctctaagcttgtcaatgaggttttatcacatgtataaccggcatcagtacccatttgtcgtccttaacgtttccgttgactcagaatgtgtggatattaatgtaactccagataaaaggcaaattctactacaagaagagaagctattgctggccgttttaaagacctccttgataggaatgtttgacagtgatgcaaacaagcttaatgtcaaccagcagccactgctagatgttgaaggtaacttagtaaagctgcatactgcagaactagaaaagcctgtgccaggaaagcaagataactctccttcactgaagagcacagcagacgagaaaagggtagcatccatctccaggctgagagaggccttttctcttcatcctactaaagagatcaagtctaggggtccagagactgctgaactgacacggagttttccaagtgagaaaaggggcgtgttatcctcttatccttcagacgtcatctcttacagaggcctccgtggctcgcaggacaaattggtgagtcccacggacagccctggtgactgtatggacagagagaaaatagaaaaagactcagggctcagcagcacctcagctggctctgaggaagagttcagcaccccagaagtggccagtagctttagcagtgactataacgtgagctccctagaagacagaccttctcaggaaaccataaactgtggtgacctggactgccgtcctccaggtacaggacagtccttgaagccagaagaccatggatatcaatgcaaagctctacctctagctcgtctgtcacccacaaatgccaagcgcttcaagacagaggaaagaccctcaaatgtcaacatttctcaaagattgcctggtcctcagagcacctcagcagctgaggtcgatgtagccataaaaatgaataagagaatcgtgctcctcgagttctctctgagttctctagctaagcgaatgaagcagttacagcacctaaaggcgcagaacaaacatgaactgagttacagaaaatttagggccaagatttgccctggagaaaaccaagcagcagaagatgaactcagaaaagagattagtaaatcgatgtttgcagagatggagatcttgggtcagtttaacctgggatttatagtaaccaaactgaaagaggacctcttcctggtggaccagcatgctgcggatgagaagtacaactttgagatgctgcagcagcacacggtgctccaggcgcagaggctcatcacaccccagactctgaacttaactgctgtcaatgaagctgtactgatagaaaatctggaaatattcagaaagaatggctttgactttgtcattgatgaggatgctccagtcactgaaagggctaaattgatttccttaccaactagtaaaaactggacctttggaccccaagatatagatgaactgatctttatgttaagtgacagccctggggtcatgtgccggccctcacgagtcagacagatgtttgcttccagagcctgtcggaagtcagtgatgattggaacggcgctcaatgcgagcgagatgaagaagctcatcacccacatgggtgagatggaccacccctggaactgcccccacggcaggccaaccatgaggcacgttgccaatctggatgtcatctctcagaactgacacaccccttgtagcatagagtttattacagattgttcggtttgcaaagagaaggttttaagtaatctgattatcgttgtacaaaaattagcatgctgctttaatgtactggatccatttaaaagcagtgttaaggcaggcatgatggagtgttcctctagctcagctacttgggtgatccggtgggagctcatgtgagcccaggactttgagaccactccgagccacattcatgagactcaattcaaggacaaaaaaaaaaagatatttttgaagccttttaaaaaaaaa

SEQ ID NO: 4 - protein, 859 aa, Mus musculus
MEQTEGVSTECAKAIKPIDGKSVHQICSGQVILSLSTAVKELIENSVDAGATTIDLRLKDYGVDLIEVSDNGCGVEEENFEGLALKHHTSKIQEFADLTQVETFGFRGEALSSLCALSDVTISTCHGSASVGTRLVFDHNGKITQKTPYPRPKGTTVSVQHLFYTLPVRYKEFQRNIKKEYSKMVQVLQAYCIISAGVRVSCTNQLGQGKRHAVVCTSGTSGMKENIGSVFGQKQLQSLIPFVQLPPSDAVCEEYGLSTSGRHKTFSTFRASFHSARTAPGGVQQTGSFSSSIRGPVTQQRSLSLSMRFYHMYNRHQYPFVVLNVSVDSECVDINVTPDKRQILLQEEKLLLAVLKTSLIGMFDSDANKLNVNQQPLLDVEGNLVKLHTAELEKPVPGKQDNSPSLKSTADEKRVASISRLREAFSLHPTKEIKSRGPETAELTRSFPSEKRGVLSSYPSDVISYRGLRGSQDKLVSPTDSPGDCMDREKIEKDSGLSSTSAGSEEEFSTPEVASSFSSDYNVSSLEDRPSQETINCGDLDCRPPGTGQSLKPEDHGYQCKALPLARLSPTNAKRFKTEERPSNVNISQRLPGPQSTSAAEVDVAIKMNKRIVLLEFSLSSLAKRMKQLQHLKAQNKHELSYRKFRAKICPGENQAAEDELRKEISKSMFAEMEILGQFNLGFIVTKLKEDLFLVDQHAADEKYNFEMLQQHTVLQAQRLITPQTLNLTAVNEAVLIENLEIFRKNGFDFVIDEDAPVTERAKLISLPTSKNWTFGPQDIDELIFMLSDSPGVMCRPSRVRQMFASRACRKSVMIGTALNASEMKKLITHMGEMDHPWNCPHGRPTMRHVANLDVISQN

SEQ ID NO: 5 - DNA, 2771 bp, Homo sapiens
cgaggcggatcgggtgttgcatccatggagcgagctgagagctcgagtacagaacctgctaaggccatcaaacctattgatcggaagtcagtccatcagatttgctctgggcaggtggtactgagtctaagcactgcggtaaaggagttagtagaaaacagtctggatgctggtgccactaatattgatctaaagcttaaggactatggagtggatcttattgaagtttcagacaatggatgtggggtagaagaagaaaacttcgaaggcttaactctgaaacatcacacatctaagattcaagagtttgccgacctaactcaggttgaaacttttggctttcggggggaagctctgagctcactttgtgcactgagcgatgtcaccatttctacctgccacgcatcggcgaaggttggaactcgactgatgtttgatcacaatgggaaaattatccagaaaaccccctacccccgccccagagggaccacagtcagcgtgcagcagttattttccacactacctgtgcgccataaggaatttcaaaggaatattaagaaggagtatgccaaaatggtccaggtcttacatgcatactgtatcatttcagcaggcatccgtgtaagttgcaccaatcagcttggacaaggaaaacgacagcctgtggtatgcacaggtggaagccccagcataaaggaaaatatcggctctgtgtttgggcagaagcagttgcaaagcctcattccttttgttcagctgccccctagtgactccgtgtgtgaagagtacggtttgagctgttcggatgctctgcataatcttttttacatctcaggtttcatttcacaatgcacgcatggagttggaaggagttcaacagacagacagtttttctttatcaaccggcggccttgtgacccagcaaaggtctgcagactcgtgaatgaggtctaccacatgtataatcgacaccagtatccatttgttgttcttaacatttctgttgattcagaatgcgttgatatcaatgttactccagataaaaggcaaattttgctacaagaggaaaagcttttgttggcagttttaaagacctctttgataggaatgtttgatagtgatgtcaacaagctaaatgtcagtcagcagccactgctggatgttgaaggtaacttaataaaaatgcatgcagcggatttggaaaagcccatggtagaaaagcaggatcaatccccttcattaaggactggagaagaaaaaaaagacgtgtccatttccagactgcgagaggccttttctcttcgtcacacaacagagaacaagcctcacagcccaaagactccagaaccaagaaggagccctctaggacagaaaaggggtatgctgtcttctagcacttcaggtgccatctctgacaaaggcgtcctgagacctcagaaagaggcagtgagttccagtcacggacccagtgaccctacggacagagcggaggtggagaaggactcggggcacggcagcacttccgtggattctgaggggttcagcatcccagacacgggcagtcactgcagcagcgagtatgcggccagctccccaggggacaggggctcgcaggaacatgtggactctcaggagaaagcgcctgaaactgacgactctttttcagatgtggactgccattcaaaccaggaagataccggatgtaaatttcgagttttgcctcagccaactaatctcgcaaccccaaacacaaagcgttttaaaaaagaagaaattctttccagttctgacatttgtcaaaagttagtaaatactcaggacatgtcagcctctcaggttgatgtagctgtgaaaattaataagaaagttgtgcccctggacttttctatgagttctttagctaaacgaataaagcagttacatcatgaagcacagcaaagtgaaggggaacagaattacaggaagtttagggcaaagatttgtcctggagaaaatcaagcagccgaagatgaactaagaaaagagataagtaaaacgatgtttgcagaaatggaaatcattggtcagtttaacctgggatttataataaccaaactgaatgaggatatcttcatagtggaccagcatgccacggacgagaagtataacttcgagatgctgcagcagcacaccgtgctccaggggcagaggctcatagcacctcagactctcaacttaactgctgttaatgaagctgttctgatagaaaatctggaaatatttagaaagaatggctttgattttgttatcgatgaaaatgctccagtcactgaaagggctaaactgatttccttgccaactagtaaaaactggaccttcggaccccaggacgtcgatgaactgatcttcatgctgagcgacagccctggggtcatgtgccggccttcccgagtcaagcagatgtttgcctccagagcctgccggaagtcggtgatgattgggactgctcttaacacaagcgagatgaagaaactgatcacccacatgggggagatggaccacccctggaactgtccccatggaaggccaaccatgagacacatcgccaacctgggtgtcatttctcagaactgaccgtagtcactgtatggaataattggttttatcgcagatttttatgttttgaaagacagagtcttcactaaccttttttgttttaaaatgaaacctgctacttaaaaaaaatacacatcacacccatttaaaagtgatcttgagaaccttttcaaacc

SEQ ID NO: 6 - protein, 932 aa, Homo sapiens
MKQLPAATVRLLSSSQIITSVVSVVKELIENSLDAGATSVDVKLENYGFDKIEVRDNGEGIKAVDAPVMAMKYYTSKINSHEDLENLTTYGFRGEALGSICCIAEVLITTRTAADNFSTQYVLDGSGHILSQKPSHLGQGTTVTALRLFKNLPVRKQFYSTAKKCKDEIKKIQDLLMSFGILKPDLRIVFVHNKAVIWQKSRVSDHKMALMSVLGTAVMNNMESFQYHSEESQIYLSGFLPKCDADHSFTSLSTPERSFIFINSRPVHQKDILKLIRHHYNLKCLKESTRLYPVFFLKIDVPTADVDVNLTPDKSQVLLQNKESVLIALENLMTTCYGPLPSTNSYENNKTDVSAADIVLSKTAETDVLFNKVESSGKNYSNVDTSVIPFQNDMHNDESGKNTDDCLNHQISIGDFGYGHCSSEISNIDKNTKNAFQDISMSNVSWENSQTEYSKTCFISSVKHTQSENGNKDHIDESGENEEEAGLENSSEISADEWSRGNILKNSVGENIEPVKILVPEKSLPCKVSNNNYPIPEQMNLNEDSCNKKSNVIDNKSGKVTAYDLLSNRVIKKPMSASALFVQDHRPQFLIENPKTSLEDATLQIEELWKTLSEEEKLKYEEKATKDLERYNSQMKRAIEQESQMSLKDGRKKIKPTSAWNLAQKHKLKTSLSNQPKLDELLQSQIEKRRSQNIKMVQIPFSMKNLKINFKKQNKVDLEEKDEPCLIHNLRFPDAWLMTSKTEVMLLNPYRVEEALLFKRLLENHKLPAEPLEKPIMLTESLFNGSHYLDVLYKMTADDQRYSGSTYLSDPRLTANGFKIKLIPGVSITENYLEIEGMANCLPFYGVADLKEILNAILNRNAKEVYECRPRKVISYLEGEAVRLSRQLPMYLSKEDIQDIIYRMKHQFGNEIKECVHGRPFFHHLTYLPETT

SEQ ID NO: 7 - DNA, 3063 bp, Homo sapiens
ggcacgagtggctgcttgcggctagtggatggtaattgcctgcctcgcgctagcagcaagctgctctgttaaaagcgaaaatgaaacaattgcctgcggcaacagttcgactcctttcaagttctcagatcatcacttcggtggtcagtgttgtaaaagagcttattgaaaactccttggatgctggtgccacaagcgtagatgttaaactggagaactatggatttgataaaattgaggtgcgagataacggggagggtatcaaggctgttgatgcacctgtaatggcaatgaagtactacacctcaaaaataaatagtcatgaagatcttgaaaatttgacaacttacggttttcgtggagaagccttggggtcaatttgttgtatagctgaggttttaattacaacaagaacggctgctgataattttagcacccagtatgttttagatggcagtggccacatactttctcagaaaccttcacatcttggtcaaggtacaactgtaactgctttaagattatttaagaatctacctgtaagaaagcagttttactcaactgcaaaaaaatgtaaagatgaaataaaaaagatccaagatctcctcatgagctttggtatccttaaacctgacttaaggattgtctttgtacataacaaggcagttatttggcagaaaagcagagtatcagatcacaagatggctctcatgtcagttctggggactgctgttatgaacaatatggaatcctttcagtaccactctgaagaatctcagatttatctcagtggatttcttccaaagtgtgatgcagaccactctttcactagtctttcaacaccagaaagaagtttcatcttcataaacagtcgaccagtacatcaaaaagatatcttaaagttaatccgacatcattacaatctgaaatgcctaaaggaatctactcgtttgtatcctgttttctttctgaaaatcgatgttcctacagctgatgttgatgtaaatttaacaccagataaaagccaagtattattacaaaataaggaatctgttttaattgctcttgaaaatctgatgacgacttgttatggaccattacctagtacaaattcttatgaaaataataaaacagatgtttccgcagctgacatcgttcttagtaaaacagcagaaacagatgtgctttttaataaagtggaatcatctggaaagaattattcaaatgttgatacttcagtcattccattccaaaatgatatgcataatgatgaatctggaaaaaacactgatgattgtttaaatcaccagataagtattggtgactttggttatggtcattgtagtagtgaaatttctaacattgataaaaacactaagaatgcatttcaggacatttcaatgagtaatgtatcatgggagaactctcagacggaatatagtaaaacttgttttataagttccgttaagcacacccagtcagaaaatggcaataaagaccatatagatgagagtggggaaaatgaggaagaagcaggtcttgaaaactcttcggaaatttctgcagatgagtggagcaggggaaatatacttaaaaattcagtgggagagaatattgaacctgtgaaaattttagtgcctgaaaaaagtttaccatgtaaagtaagtaataataattatccaatccctgaacaaatgaatcttaatgaagattcatgtaacaaaaaatcaaatgtaatagataataaatctggaaaagttacagcttatgatttacttagcaatcgagtaatcaagaaacccatgtcagcaagtgctctttttgttcaagatcatcgtcctcagtttctcatagaaaatcctaagactagtttagaggatgcaacactacaaattgaagaactgtggaagacattgagtgaagaggaaaaactgaaatatgaagagaaggctactaaagacttggaacgatacaatagtcaaatgaagagagccattgaacaggagtcacaaatgtcactaaaagatggcagaaaaaagataaaacccaccagcgcatggaatttggcccagaagcacaagttaaaaacctcattatctaatcaaccaaaacttgatgaactccttcagtcccaaattgaaaaaagaaggagtcaaaatattaaaatggtacagatccccttttctatgaaaaacttaaaaataaattttaagaaacaaaacaaagttgacttagaagagaaggatgaaccttgcttgatccacaatctcaggtttcctgatgcatggctaatgacatccaaaacagaggtaatgttattaaatccatatagagtagaagaagccctgctatttaaaagacttcttgagaatcataaacttcctgcagagccactggaaaagccaattatgttaacagagagtctttttaatggatctcattatttagacgttttatataaaatgacagcagatgaccaaagatacagtggatcaacttacctgtctgatcctcgtcttacagcgaatggtttcaagataaaattgataccaggagtttcaattactgaaaattacttggaaatagaaggaatggctaattgtctcccattctatggagtagcagatttaaaagaaattcttaatgctatattaaacagaaatgcaaaggaagtttatgaatgtagacctcgcaaagtgataagttatttagagggagaagcagtgcgtctatccagacaattacccatgtacttatcaaaagaggacatccaagacattatctacagaatgaagcaccagtttggaaatgaaattaaagagtgtgttcatggtcgcccattttttcatcatttaacctatcttccagaaactacatgattaaatatgtttaagaagattagttaccattgaaattggttctgtcataaaacagcatgagtctggttttaaattatctttgtattatgtgtcacatggttattttttaaatgaggattcactgacttgtttttatattgaaaaaagttccacgtattgtagaaaacgtaaataaactaataac

SEQ ID NO: 8 - protein, 932 aa, Homo sapiens; identical to SEQ ID NO: 6

SEQ ID NO: 9 - DNA, 3145 bp, Homo sapiens
ggcgggaaacagcttagtgggtgtggggtcgcgcattttcttcaaccaggaggtgaggaggtttcgacatggcggtgcagccgaaggagacgctgcagttggagagcgcggccgaggtcggcttcgtgcgcttctttcagggcatgccggagaagccgaccaccacagtgcgccttttcgaccggggcgacttctatacggcgcacggcgaggacgcgctgctggccgcccgggaggtgttcaagacccagggggtgatcaagtacatggggccggcaggagcaaagaatctgcagagtgttgtgcttagtaaaatgaattttgaatcttttgtaaaagatcttcttctggttcgtcagtatagagttgaagtttataagaatagagctggaaataaggcatccaaggagaatgattggtatttggcatataaggcttctcctggcaatctctctcagtttgaagacattctctttggtaacaatgatatgtcagcttccattggtgttgtgggtgttaaaatgtccgcagttgatggccagagacaggttggagttgggtatgtggattccatacagaggaaactaggactgtgtgaattccctgataatgatcagttctccaatcttgaggctctcctcatccagattggaccaaaggaatgtgttttacccggaggagagactgctggagacatggggaaactgagacagataattcaaagaggaggaattctgatcacagaaagaaaaaaagctgacttttccacaaaagacatttatcaggacctcaaccggttgttgaaaggcaaaaagggagagcagatgaatagtgctgtattgccagaaatggagaatcaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcagggttctgttgaagataccactggctctcagtctctggctgccttgctgaataagtgtaaaacccctcaaggacaaagacttgttaaccagtggattaagcagcctctcatggataagaacagaatagaggagagattgaatttagtggaagcttttgtagaagatgcagaattgaggcagactttacaagaagatttacttcgtcgattcccagatcttaaccgacttgccaagaagtttcaaagacaagcagcaaacttacaagattgttaccgactctatcagggtataaatcaactacctaatgttatacaggctctggaaaaacatgaaggaaaacaccagaaattattgttggcagtttttgtgactcctcttactgatcttcgttctgacttctccaagtttcaggaaatgatagaaacaactttagatatggatcaggtggaaaaccatgaattccttgtaaaaccttcatttgatcctaatctcagtgaattaagagaaataatgaatgacttggaaaagaagatgcagtcaacattaataagtgcagccagagatcttggcttggaccctggcaaacagattaaactggattccagtgcacagtttggatattactttcgtgtaacctgtaaggaagaaaaagtccttcgtaacaataaaaactttagtactgtagatatccagaagaatggtgttaaatttaccaacagcaaattgacttctttaaatgaagagtataccaaaaataaaacagaatatgaagaagcccaggatgccattgttaaagaaattgtcaatatttcttcaggctatgtagaaccaatgcagacactcaatgatgtgttagctcagctagatgctgttgtcagctttgctcacgtgtcaaatggagcacctgttccatatgtacgaccagccattttggagaaaggacaaggaagaattatattaaaagcatccaggcatgcttgtgttgaagttcaagatgaaattgcatttattcctaatgacgtatactttgaaaaagataaacagatgttccacatcattactggccccaatatgggaggtaaatcaacatatattcgacaaactggggtgatagtactcatggcccaaattgggtgttttgtgccatgtgagtcagcagaagtgtccattgtggactgcatcttagcccgagtaggggctggtgacagtcaattgaaaggagtctccacgttcatggctgaaatgttggaaactgcttctatcctcaggtctgcaaccaaagattcattaataatcatagatgaattgggaagaggaacttctacctacgatggatttgggttagcatgggctatatcagaatacattgcaacaaagattggtgctttttgcatgtttgcaacccattttcatgaacttactgccttggccaatcagataccaactgttaataatctacatgtcacagcactcaccactgaagagaccttaactatgctttatcaggtgaagaaaggtgtctgtgatcaaagttttgggattcatgttgcagagcttgctaatttccctaagcatgtaatagagtgtgctaaacagaaagccctggaacttgaggagtttcagtatattggagaatcgcaaggatatgatatcatggaaccagcagcaaagaagtgctatctggaaagagagcaaggtgaaaaaattattcaggagttcctgtccaaggtgaaacaaatgccctttactgaaatgtcagaagaaaacatcacaataaagttaaaacagctaaaagctgaagtaatagcaaagaataatagctttgtaaatgaaatcatttcacgaataaaagttactacgtgaaaaatcccagtaatggaatgaaggtaatattgataagctattgtctgtaatagttttatattgttttatattaaccctttttccatagtgttaactgtcagtgcccatgggctatcaacttaataagatatttagtaatattttactttgaggacattttcaaagatttttattttgaaaaatgagagctgtaactgaggactgtttgcaattgacataggcaataataagtgatgtgctgaattttataaataaaatcatgtagtttgtgg

SEQ ID NO: 10 - protein, 934 aa, Homo sapiens [sequence omitted]

SEQ ID NO: 11 - DNA, 2484 bp, Homo sapiens
cttggctcttctggcgccaaaatgtcgttcgtggcaggggttattcggcggctggacgagacagtggtgaaccgcatcgcggcgggggaagttatccagcggccagctaatgctatcaaagagatgattgagaactgtttagatgcaaaatccacaagtattcaagtgattgttaaagagggaggcctgaagttgattcagatccaagacaatggcaccgggatcaggaaagaagatctggatattgtatgtgaaaggttcactactagtaaactgcagtcctttgaggatttagccagtatttctacctatggctttcgaggtgaggctttggccagcataagccatgtggctcatgttactattacaacgaaaacagctgatggaaagtgtgcatacagagcaagttactcagatggaaaactgaaagcccctcctaaaccatgtgctggcaatcaagggacccagatcacggtggaggaccttttttacaacatagccacgaggagaaaagctttaaaaaatccaagtgaagaatatgggaaaattttggaagttgttggcaggtattcagtacacaatgcaggcattagtttctcagttaaaaaacaaggagagacagtagctgatgttaggacactacccaatgcctcaaccgtggacaatattcgctccatctttggaaatgctgttagtcgagaactgatagaaattggatgtgaggataaaaccctagccttcaaaatgaatggttacatatccaatgcaaactactcagtgaagaagtgcatcttcttactcttcatcaaccatcgtctggtagaatcaacttccttgagaaaagccatagaaacagtgtatgcagcctatttgcccaaaaacacacacccattcctgtacctcagtttagaaatcagtccccagaatgtggatgttaatgtgcaccccacaaagcatgaagttcacttcctgcacgaggagagcatcctggagcgggtgcagcagcacatcgagagcaagctcctgggctccaattcctccaggatgtacttcacccagactttgctaccaggacttgctggcccctctggggagatggttaaatccacaacaagtctgacctcgtcttctacttctggaagtagtgataaggtctatgcccaccagatggttcgtacagattcccgggaacagaagcttgatgcatttctgcagcctctgagcaaacccctgtccagtcagccccaggccattgtcacagaggataagacagatatttctagtggcagggctaggcagcaagatgaggagatgcttgaactcccagcccctgctgaagtggctgccaaaaatcagagcttggagggggatacaacaaaggggacttcagaaatgtcagagaagagaggacctacttccagcaaccccagaaagagacatcgggaagattctgatgtggaaatggtggaagatgattcccgaaaggaaatgactgcagcttgtaccccccggagaaggatcattaacctcactagtgttttgagtctccaggaagaaattaatgagcagggacatgaggttctccgggagatgttgcataaccactccttcgtgggctgtgtgaatcctcagtgggccttggcacagcatcaaaccaagttataccttctcaacaccaccaagcttagtgaagaactgttctaccagatactcatttatgattttgccaattttggtgttctcaggttatcggagccagcaccgctctttgaccttgccatgcttgccttagatagtccagagagtggctggacagaggaagatggtcccaaagaaggacttgctgaatacattgttgagtttctgaagaagaaggctgagatgcttgcagactatttctctttggaaattgatgaggaagggaacctgattggattaccccttctgattgacaactatgtgccccctttggagggactgcctatcttcattcttcgactagccactgaggtgaattgggacgaagaaaaggaatgttttgaaagcctcagtaaagaatgcgctatgttctattccatccggaagcagtacatatctgaggagtcgaccctctcaggccagcagagtgaagtgcctggctccattccaaactcctggaagtggactgtggaacacattgtctataaagccttgcgctcacacattctgcctcctaaacatttcacagaagatggaaatatcctgcagcttgctaacctgcctgatctatacaaagtctttgagaggtgttaaatatggttatttatgcactgtgggatgtgttcttctttctctgtattccgatacaaagtgttgtatcaaagtgtgatatacaaagtgtaccaacataagtgttggtagcacttaagacttatacttgccttctgatagtattcctttatacacagtggattgattataaataaatagatgtgtcttaacata

SEQ ID NO: 12 - protein, 756 aa, Homo sapiens
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 13 - DNA, 426 bp, Homo sapiens
cgaggcggatcgggtgttgcatccatggagcgagctgagagctcgagtacagaacctgctaaggccatcaaacctattgatcggaagtcagtccatcagatttgctctgggcaggtggtactgagtctaagcactgcggtaaaggagttagtagaaaacagtctggatgctggtgccactaatattgatctaaagcttaaggactatggagtggatcttattgaagtttcagacaatggatgtggggtagaagaagaaaacttcgaaggcttaactctgaaacatcacacatctaagattcaagagtttgccgacctaactcaggttgaaacttttggctttcggggggaagctctgagctcactttgtgcactgagcgatgtcaccatttctacctgccacgcatcggcgaaggttggaacttga

SEQ ID NO: 14 - protein, 133 aa, Homo sapiens
MKQLPAATVRLLSSSQIITSVVSVVKELIENSLDAGATSVDVKLENYGFDKIEVRDNGEGIKAVDAPVMAMKYYTSKINSHEDLENLTTYGFRGEALGSICCIAEVLITTRTAADNFSTQYVLDGSGHILSQK

SEQ ID NO: 15 - DNA, 4264 bp, Homo sapiens
atttcccgccagcaggagccgcgcggtagatgcggtgcttttaggagctccgtccgacagaacggttgggccttgccggctgtcggtatgtcgcgacagagcaccctgtacagcttcttccccaagtctccggcgctgagtgatgccaacaaggcctcggccagggcctcacgcgaaggcggccgtgccgccgctgcccccggggcctctccttccccaggcggggatgcggcctggagcgaggctgggcctgggcccaggcccttggcgcgatccgcgtcaccgcccaaggcgaagaacctcaacggagggctgcggagatcggtagcgcctgctgcccccaccagttgtgacttctcaccaggagatttggtttgggccaagatggagggttacccctggtggccttgtctggtttacaaccacccctttgatggaacattcatccgcgagaaagggaaatcagtccgtgttcatgtacagttttttgatgacagcccaacaaggggctgggttagcaaaaggcttttaaagccatatacaggttcaaaatcaaaggaagcccagaagggaggtcatttttacagtgcaaagcctgaaatactgagagcaatgcaacgtgcagatgaagccttaaataaagacaagattaagaggcttgaattggcagtttgtgatgagccctcagagccagaagaggaagaagagatggaggtaggcacaacttacgtaacagataagagtgaagaagataatgaaattgagagtgaagaggaagtacagcctaagacacaaggatctaggcgaagtagccgccaaataaaaaaacgaagggtcatatcagattctgagagtgacattggtggctctgatgtggaatttaagccagacactaaggaggaaggaagcagtgatgaaataagcagtggagtgggggatagtgagagtgaaggcctgaacagccctgtcaaagttgctcgaaagcggaagagaatggtgactggaaatggctctcttaaaaggaaaagctctaggaaggaaacgccctcagccaccaaacaagcaactagcatttcatcagaaaccaagaatactttgagagctttctctgcccctcaaaattctgaatcccaagcccacgttagtggaggtggtgatgacagtagtcgccctactgtttggtatcatgaaactttagaatggcttaaggaggaaaagagaagagatgagcacaggaggaggcctgatcaccccgattttgatgcatctacactctatgtgcctgaggatttcctcaattcttgtactcctgggatgaggaagtggtggcagattaagtctcagaactttgatcttgtcatctgttacaaggtggggaaattttatgagctgtaccacatggatgctcttattggagtcagtgaactggggctggtattcatgaaaggcaactgggcccattctggctttcctgaaattgcatttggccgttattcagattccctggtgcagaagggctataaagtagcacgagtggaacagactgagactccagaaatgatggaggcacgatgtagaaagatggcacatatatccaagtatgatagagtggtgaggagggagatctgtaggatcattaccaagggtacacagacttacagtgtgctggaaggtgatccctctgagaactacagtaagtatcttcttagcctcaaagaaaaagaggaagattcttctggccatactcgtgcatatggtgtgtgctttgttgatacttcactgggaaagtttttcataggtcagttttcagatgatcgccattgttcgagatttaggactctagtggcacactatcccccagtacaagttttatttgaaaaaggaaatctctcaaaggaaactaaaacaattctaaagagttcattgtcctgttctcttcaggaaggtctgatacccggctcccagttttgggatgcatccaaaactttgagaactctccttgaggaagaatattttagggaaaagctaagtgatggcattggggtgatgttaccccaggtgcttaaaggtatgacttcagagtctgattccattgggttgacaccaggagagaaaagtgaattggccctctctgctctaggtggttgtgtcttctacctcaaaaaatgccttattgatcaggagcttttatcaatggctaattttgaagaatatattcccttggattctgacacagtcagcactacaagatctggtgctatcttcaccaaagcctatcaacgaatggtgctagatgcagtgacattaaacaacttggagatttttctgaatggaacaaatggttctactgaaggaaccctactagagagggttgatacttgccatactccttttggtaagcggctcctaaagcaatggctttgtgccccactctgtaaccattatgctattaatgatcgtctagatgccatagaagacctcatggttgtgcctgacaaaatctccgaagttgtagagcttctaaagaagcttccagatcttgagaggctactcagtaaaattcataatgttgggtctcccctgaagagtcagaaccacccagacagcagggctataatgtatgaagaaactacatacagcaagaagaagattattgattttctttctgctctggaaggattcaaagtaatgtgtaaaattatagggatcatggaagaagttgctgatggttttaagtctaaaatccttaagcaggtcatctctctgcagacaaaaaatcctgaaggtcgttttcctgatttgactgtagaattgaaccgatgggatacagcctttgaccatgaaaaggctcgaaagactggacttattactcccaaagcaggctttgactctgattatgaccaagctcttgctgacataagagaaaatgaacagagcctcctggaatacctagagaaacagcgcaacagaattggctgtaggaccatagtctattgggggattggtaggaaccgttaccagctggaaattcctgagaatttcaccactcgcaatttgccagaagaatacgagttgaaatctaccaagaagggctgtaaacgatactggaccaaaactattgaaaagaagttggctaatctcataaatgctgaagaacggagggatgtatcattgaaggactgcatgcggcgactgttctataactttgataaaaattacaaggactggcagtctgctgtagagtgtatcgcagtgttggatgttttactgtgcctggctaactatagtcgagggggtgatggtcctatgtgtcgcccagtaattctgttgccggaagataccccccccttcttagagcttaaaggatcacgccatccttgcattacgaagactttttttggagatgattttattcctaatgacattctaataggctgtgaggaagaggagcaggaaaatggcaaagcctattgtgtgcttgttactggaccaaatatggggggcaagtctacgcttatgagacaggctggcttattagctgtaatggcccagatgggttgttacgtccctgctgaagtgtgcaggctcacaccaattgatagagtgtttactagacttggtgcctcagacagaataatgtcaggtgaaagtacattttttgttgaattaagtgaaactgccagcatactcatgcatgcaacagcacattctctggtgcttgtggatgaattaggaagaggtactgcaacatttgatgggacggcaatagcaaatgcagttgttaaagaacttgctgagactataaaatgtcgtacattattttcaactcactaccattcattagtagaagattattctcaaaatgttgctgtgcgcctaggacatatggcatgcatggtagaaaatgaatgtgaagaccccagccaggagactattacgttcctctataaattcattaagggagcttgtcctaaaagctatggctttaatgcagcaaggcttgctaatctcccagaggaagttattcaaaagggacatagaaaagcaagagaatttgagaagatgaatcagtcactacgattatttcgggaagtttgcctggctagtgaaaggtcaactgtagatgctgaagctgtccataaattgctgactttgattaaggaattatagactgactacattggaagctttgagttgacttctgaccaaaggtggtaaattcagacaacattatgatctaataaactttattttttaaaaatga

SEQ ID NO: 16 - protein, 1360 aa, Homo sapiens
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGKSTLMRQAGLLAVMAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKEL

SEQ ID NO: 17 - DNA, 1408 bp, Homo sapiens
ggcgctcctacctgcaagtggctagtgccaagtgctgggccgccgctcctgccgtgcatgttggggagccagtacatgcaggtgggctccacacggagaggggcgcagacccggtgacagggctttacctggtacatcggcatggcgcaaccaaagcaagagagggtggcgcgtgccagacaccaacggtcggaaaccgccagacaccaacggtcggaaaccgccaagacaccaacgctcggaaaccgccagacaccaacgctcggaaaccgccagacaccaaggctcggaatccacgccaggccacgacggagggcgactacctcccttctgaccctgctgctggcgttcggaaaaaacgcagtccggtgtgctctgattggtccaggctctttgacgtcacggactcgacctttgacagagccactaggcgaaaaggagagacgggaagtattttttccgccccgcccggaaagggtggagcacaacgtcgaaagcagccgttgggagcccaggaggcggggcgcctgtgggagccgtggagggaactttcccagtccccgaggcggatccggtgttgcatccttggagcgagctgagaactcgagtacagaacctgctaaggccatcaaacctattgatcggaagtcagtccatcagatttgctctgggccggtggtaccgagtctaaggccgaatgcggtgaaggagttagtagaaaacagtctggatgctggtgccactaatgttgatctaaagcttaaggactatggagtggatctcattgaagtttcaggcaatggatgtggggtagaagaagaaaacttcgaaggctttactctgaaacatcacacatgtaagattcaagagtttgccgacctaactcaggtggaaacttttggctttcggggggaagctctgagctcactttgtgcactgagtgatgtcaccatttctacctgccgtgtatcagcgaaggttgggactcgactggtgtttgatcactatgggaaaatcatccagaaaaccccctacccccgccccagagggatgacagtcagcgtgaagcagttattttctacgctacctgtgcaccataaagaatttcaaaggaatattaagaagaaacgtgcctgcttccccttcgccttctgccgtgattgtcagtttcctgaggcctccccagccatgcttcctgtacagcctgtagaactgactcctagaagtaccccaccccacccctgctccttggaggacaacgtgatcactgtattcagctctgtcaagaatggtccaggttcttctagatgatctgcacaaatggttcctctcctccttcctgatgtctgccattagcattggaataaagttcctgctgaaaatccaaaaaaaaaaaaaaaaaaaaaaa

SEQ ID NO: 18 - protein, 389 aa, Homo sapiens
MAQPKQERVARARHQRSETARHQRSETAKTPTLGNRQTPTLGNRQTPRLGIHARPRRRATTSLLTLLLAFGKNAVRCALIGPGSLTSRTRPLTEPLGEKERREVFFPPRPERVEHNVESSRWEPRRRGACGSRGGNFPSPRGGSGVASLERAENSSTEPAKAIKPIDRKSVHQICSGPVVPSLRPNAVKELVENSLDAGATNVDLKLKDYGVDLIEVSGNGCGVEEENFEGFTLKHHTCKIQEFADLTQVETFGFRGEALSSLCALSDVTISTCRVSAKVGTRLVFDHYGKIIQKTPYPRPRGMTVSVKQLFSTLPVHHKEFQRNIKKKRACFPFAFCRDCQFPEASPAMLPVQPVELTPRSTPPHPCSLEDNVITVFSSVKNGPGSSR

SEQ ID NO: 19 - DNA, 1785 bp, Homo sapiens
tttttagaaactgatgtttattttccatcaaccatttttccatgctgcttaagagaatatgcaagaacagcttaagaccagtcagtggttgctcctacccattcagtggcctgagcagtggggagctgcagaccagtcttccgtggcaggctgagcgctccagtcttcagtagggaattgctgaataggcacagagggcacctgtacaccttcagaccagtctgcaacctcaggctgagtagcagtgaactcaggagcgggagcagtccattcaccctgaaattcctccttggtcactgccttctcagcagcagcctgctcttctttttcaatctcttcaggatctctgtagaagtacagatcaggcatgacctcccatgggtgttcacgggaaatggtgccacgcatgcgcagaacttcccgagccagcatccaccacattaaacccactgagtgagctcccttgttgttgcatgggatggcaatgtccacatagcgcagaggagaatctgtgttacacagcgcaatggtaggtaggttaacataagatgcctccgtgagaggcgaaggggcggcgggacccgggcctggcccgtatgtgtccttggcggcctagactaggccgtcgctgtatggtgagccccagggaggcggatctgggcccccagaaggacacccgcctggatttgccccgtagcccggcccgggcccctcgggagcagaacagccttggtgaggtggacaggaggggacctcgcgagcagacgcgcgcgccagcgacagcagccccgccccggcctctcgggagccggggggcagaggctgcggagccccaggagggtctatcagccacagtctctgcatgtttccaagagcaacaggaaatgaacacattgcaggggccagtgtcattcaaagatgtggctgtggatttcacccaggaggagtggcggcaactggaccctgatgagaagatagcatacggggatgtgatgttggagaactacagccatctagtttctgtggggtatgattatcaccaagccaaacatcatcatggagtggaggtgaaggaagtggagcagggagaggagccgtggataatggaaggtgaatttccatgtcaacatagtccagaacctgctaaggccatcaaacctattgatcggaagtcagtccatcagatttgctctgggccagtggtactgagtctaagcactgcagtgaaggagttagtagaaaacagtctggatgctggtgccactaatattgatctaaagcttaaggactatggagtggatctcattgaagtttcagacaatggatgtggggtagaagaagaaaactttgaaggcttaatctctttcagctctgaaacatcacacatgtaagattcaagagtttgccgacctaactgaagttgaaactttcggttttcagggggaagctctgagctcactgtgtgcactgagcgatgtcaccatttctacctgccacgcgttggtgaaggttgggactcgactggtgtttgatcacgatgggaaaatcatccaggaaaccccctacccccaccccagagggaccacagtcagcgtgaagcagttattttctacgctacctgtgcgccataaggaatttcaaaggaatattaagaagacgtgcctgcttccccttcgccttctgccgtgattgtcagtttcctgaggcctccccagccatgcttcctgtacagcctgcagaactgtgagtcaattaaacctcttttcttcataaattaaaaaaaaa

SEQ ID NO: 20 - protein, 264 aa, Homo sapiens
MCPWRPRLGRRCMVSPREADLGPQKDTRLDLPRSPARAPREQNSLGEVDRRGPREQTRAPATAAPPRPLGSRGAEAAEPQEGLSATVSACFQEQQEMNTLQGPVSFKDVAVDFTQEEWRQLDPDEKIAYGDVMLENYSHLVSVGYDYHQAKHHHGVEVKEVEQGEEPWIMEGEFPCQHSPEPAKAIKPIDRKSVHQICSGPVVLSLSTAVKELVENSLDAGATNIDLKLKDYGVDLIEVSDNGCGVEEENFEGLISFSSETSHM

SEQ ID NO: 21 - DNA, 795 bp, Homo sapiens
atgtgtccttggcggcctagactaggccgtcgctgtatggtgagccccagggaggcggatctgggcccccagaaggacacccgcctggatttgccccgtagcccggcccgggcccctcgggagcagaacagccttggtgaggtggacaggaggggacctcgcgagcagacgcgcgcgccagcgacagcagccccgccccggcctctcgggagccggggggcagaggctgcggagccccaggagggtctatcagccacagtctctgcatgtttccaagagcaacaggaaatgaacacattgcaggggccagtgtcattcaaagatgtggctgtggatttcacccaggaggagtggcggcaactggaccctgatgagaagatagcatacggggatgtgatgttggagaactacagccatctagtttctgtggggtatgattatcaccaagccaaacatcatcatggagtggaggtgaaggaagtggagcagggagaggagccgtggataatggaaggtgaatttccatgtcaacatagtccagaacctgctaaggccatcaaacctattgatcggaagtcagtccatcagatttgctctgggccagtggtactgagtctaagcactgcagtgaaggagttagtagaaaacagtctggatgctggtgccactaatattgatctaaagcttaaggactatggagtggatctcattgaagtttcagacaatggatgtggggtagaagaagaaaactttgaaggcttaatctctttcagctctgaaacatcacacatgtaa

SEQ ID NO: 22 - protein, 264 aa, Homo sapiens; identical to SEQ ID NO: 20

SEQ ID NO: 23 - DNA, 30 bp, Artificial Sequence
  synthetic oligonucleotide primer
acgcatatggagcgagctgagagctcgagt

SEQ ID NO: 24 - DNA, 75 bp, Artificial Sequence
  synthetic oligonucleotide primer
gaattcttatcacgtagaatcgagaccgaggagagggttagggataggcttaccagttccaaccttcgccgatgc

SEQ ID NO: 25 - DNA, 27 bp, Artificial Sequence
  synthetic oligonucleotide primer
acgcatatgtgtccttggcggcctaga

SEQ ID NO: 26 - DNA, 75 bp, Artificial Sequence
  synthetic oligonucleotide primer
gaattcttattacgtagaatcgagaccgaggagagggttagggataggcttacccatgtgtgatgtttcagagct

SEQ ID NO: 27 - DNA, 49 bp, Artificial Sequence
  synthetic oligonucleotide primer
tttaatacgactcactatagggagaccaccatggnnnnnnnnnnnnnnn

SEQ ID NO: 28 - DNA, 4290 bp, Homo sapiens
atgatcaagtgcttgtcagttgaagtacaagccaaattgcgttctggtttggccataagctccttgggccaatgtgttgaggaacttgccctcaacagtattgatgctgaagcaaaatgtgtggctgtcagggtgaatatggaaaccttccaagttcaagtgatagacaatggatttgggatggggagtgatgatgtagagaaagtgggaaatcgttatttcaccagtaaatgccactcggtacaggacttggagaatccaaggttttatggtttccgaggagaggccttggcaaatattgctgacatggccagtgctgtggaaatttcgtccaagaaaaacaggacaatgaaaacttttgtgaaactgtttcagagtggaaaagccctgaaagcttgtgaagctgatgtgactagagcaagcgctgggactactgtaacagtgtataacctattttaccagcttcctgtaaggaggaaatgcatggaccctagactggagtttgagaaggttaggcagagaatagaagctctctcactcatgcacccttccatttctttctctttgagaaatgatgtttctggttccatggttcttcagctccctaaaaccaaagacgtatgttcccgattttgtcaaatttatggattgggaaagtcccaaaagctaagagaaataagttttaaatataaagagtttgagcttagtggctatatcagctctgaagcacattacaacaagaatatgcagtttttgtttgtgaacaaaagactagttttaaggacaaagctacataaactcattgactttttattaaggaaagaaagtattatatgcaagccaaagaatggtcccaccagtaggcaaatgaattcaagtcttcggcaccggtctaccccagaactctatggcatatatgtaattaatgtgcagtgccaattctgtgagtatgatgtgtgcatggagccagccaaaactctgattgaatttcagaactgggacactctcttgttttgcattcaggaaggagtgaaaatgtttttaaagcaagaaaaattatttgtggaattatcaggtgaggatattaaggaatttagtgaagataatggttttagtttatttgatgctactcttcagaagcgtgtgacttccgatgagaggagcaatttccaggaagcatgtaataatattttagattcctatgagatgtttaatttgcagtcaaaagctgtgaaaagaaaaactactgcagaaaacgtaaacacacagagttctagggattcagaagctaccagaaaaaatacaaatgatgcatttttgtacatttatgaatcaggtggtccaggccatagcaaaatgacagagccatctttacaaaacaaagacagctcttgctcagaatcaaagatgttagaacaagagacaattgtagcatcagaagctggagaaaatgagaaacataaaaaatctttcctggaacatagctctttagaaaatccgtgtggaaccagtttagaaatgtttttaagcccttttcagacaccatgtcactttgaggagagtgggcaggatctagaaatatggaaagaaagtactactgttaatggcatggctgccaacatcttgaaaaataatagaattcagaatcaaccaaagagatttaaagatgctactgaagtgggatgccagcctctgccttttgcaacaacattatggggagtacatagtgctcagacagagaaagagaaaaaaaaagaatctagcaattgtggaagaagaaatgtttttagttatgggcgagttaaattatgttccactggctttataactcatgtagtacaaaatgaaaaaactaaatcaactgaaacagaacattcatttaaaaattatgttagacctggtcccacacgtgcccaagaaacatttggaaatagaacacgtcattcagttgaaactccagacatcaaagatttagccagcactttaagtaaagaatctggtcaattgcccaacaaaaaaaattgcagaacgaatataagttatgggctagagaatgaacctacagcaacttatacaatgttttctgcttttcaggaaggtagcaaaaaatcacaaacagattgcatattatctgatacatccccctctttcccctggtatagacacgtttccaatgatagtaggaaaacagataaattaattggtttctccaaaccaatcgtccgtaagaagctaagcttgagttcacagctaggatctttagagaagtttaagaggcaatatgggaaggttgaaaatcctctggatacagaagtagaggaaagtaatggagtcactaccaatctcagtcttcaagttgaacctgacattctgctgaaggacaagaaccgcttagagaactctgatgtttgtaaaatcactactatggagcatagtgattcagatagtagttgtcaaccagcaagccacatccttgactcagagaagtttccattctccaaggatgaagattgtttagaacaacagatgcttagtttgagagaaagtcctatgaccctgaaggagttatctctctttaatagaaaacctttggaccttgagaagtcatctgaatcactagcctctaaattatccagactgaagggttccgaaagagaaactcaaacaatggggatgatgagtcgttttaatgaacttccaaattcagattccagtaggaaagacagcaagttgtgcagtgtgttaacacaagatttttgtatgttatttaacaacaagcatgaaaaaacagagaatggtgtcatcccaacatcagattctgccacacaggataattcctttaataaaaatagtaaaacacattctaacagcaatacaacagagaactgtgtgatatcagaaactcctttggtattgccctataataattctaaagttaccggtaaagattcagatgttcttatcagagcctcagaacaacagataggaagtcttgactctcccagtggaatgttaatgaatccggtagaagatgccacaggtgaccaaaatggaatttgttttcagagtgaggaatctaaagcaagagcttgttctgaaactgaagagtcaaacacgtgttgttcagattggcagcggcatttcgatgtagccctgggaagaatggtttatgtcaacaaaatgactggactcagcacattcattgccccaactgaggacattcaggctgcttgtactaaagacctgacaactgtggctgtggatgttgtacttgagaatgggtctcagtacaggtgtcaaccttttagaagcgaccttgttcttcctttccttccgagagctcgagcagagaggactgtgatgagacaggataacagagatactgtggatgatactgttagtagcgaatcgcttcagtctttgttctcagaatgggacaatccagtatttgcccgttatccagaggttgctgttgatgtaagcagtggccaggctgagagcttagcagttaaaattcacaacatcttgtatccctatcgtttcaccaaaggaatgattcattcaatgcaggttctccagcaagtagataacaagtttattgcctgtttgatgagcactaagactgaagagaatggcgaggcagattcctacgagaagcaacaggcacaaggctctggtcggaaaaaattactgtcttctactctaattcctccgctagagataacagtgacagaggaacaaaggagactcttatggtgttaccacaaaaatctggaagatctgggccttgaatttgtatttccagacactagtgattctctggtccttgtgggaaaagtaccactatgttttgtggaaagagaagccaatgaacttcggagaggaagatctactgtgaccaagagtattgtggaggaatttatccgagaacaactggagctactccagaccaccggaggcatccaagggacattgccactgactgtccagaaggtgttggcatcccaagcctgccatggggccattaagtttaatgatggcctgagcttacaggaaagttgccgccttattgaagctctgtcctcatgccagctgccattccagtgtgctcacgggagaccttctatgctgccgttagctgacatagaccacttggaacaggaaaaacagattaaacccaacctcactaaacttcgcaaaatggcccaggcctggcgtctctttggaaaagcagagtgtgatacaaggcagagcctgcagcaatccatgcctccctgtgagccaccatga

SEQ ID NO: 29 - protein, 1429 aa, Homo sapiens [sequence omitted]

SEQ ID NO: 30 - DNA, 2340 bp, Arabidopsis thaliana
atgcaaggagattcttctccgtctccgacgactactagctctcctttgataagacctataaacagaaacgtaattcacagaatctgttccggtcaagtcatcttagacctctcttcggccgtcaaggagcttgtcgagaatagtctcgacgccggcgccaccagtatagagattaacctccgagactacggcgaagactattttcaggtcattgacaatggttgtggcatttccccaaccaatttcaaggtttgtgtccaaattctccgaagaacttttgatgttcttgcacttaagcatcatacttctaaattagaggatttcacagatcttttgaatttgactacttatggttttagaggagaagccttgagctctctctgtgcattgggaaatctcactgtggaaacaagaacaaagaatgagccagttgctacgctcttgacgtttgatcattctggtttgcttactgctgaaaagaagactgctcgccaaattggtaccactgtcactgttaggaagttgttctctaatttacctgtacgaagcaaagagtttaagcggaatatacgcaaagaatatgggaagcttgtatctttattgaacgcatatgcgcttattgcgaaaggagtgcggtttgtctgctctaacacgactgggaaaaacccaaagtctgttgtgctgaacacacaagggaggggttcacttaaagataatatcataacagttttcggcattagtacctttacaagtctacagcctggtactggacgcaatttagcagatcgacagtatttctttataaatggtcggcctgtagatatgccaaaagtcagcaagttggtgaatgagttatataaagatacaagttctcggaaatatccagttaccattctggattttattgtgcctggtggagcatgtgatttgaatgtcacgcccgataaaagaaaggtgttcttttctgacgagacttctgttatcggttctttgagggaaggtctgaacgagatatattcctccagtaatgcgtcttatattgttaataggttcgaggagaattcggagcaaccagataaggctggagtttcgtcgtttcagaagaaatcaaatcttttgtcagaagggatagttctggatgtcagttctaaaacaagactaggggaagctattgagaaagaaaatccatccttaagggaggttgaaattgataatagttcgccaatggagaagtttaagtttgagatcaaggcatgtgggacgaagaaaggggaaggttctttatcagtccatgatgtaactcaccttgacaagacacctagcaaaggtttgcctcagttaaatgtgactgagaaagttactgatgcaagtaaagacttgagcagccgctctagctttgcccagtcaactttgaatacttttgttaccatgggaaaaagaaaacatgaaaacataagcaccatcctctctgaaacacctgtcctcagaaaccaaacttctagttatcgtgtggagaaaagcaaatttgaagttcgtgccttagcttcaaggtgtctcgtggaaggcgatcaacttgatgatatggtcatctcaaaggaagatatgacaccaagcgaaagagattctgaactaggcaatcggatttctcctggaacacaagctgataatgttgaaagacatgagagagtactcgggcaattcaatcttgggttcatcattgcaaaattggagcgagatctgttcattgtggatcagcatgcagctgatgagaaattcaacttcgaacatttagcaaggtcaactgtcctgaaccagcaacccttactccagcctttgaacttggaactctctccagaagaagaagtaactgtgttaatgcacatggatattatcagggaaaatggctttcttctagaggagaatccaagtgctcctcccggaaaacactttagactacgagccattccttatagcaagaatatcacctttggagtcgaagatcttaaagacctgatctcaactctaggagataaccatggggaatgttcggttgctagtagctacaaaaccagcaaaacagattcgatttgtccatcacgagtccgtgcaatgctagcatcccgagcatgcagatcatctgtgatgatcggagatccactcagaaaaaacgaaatgcagaagatagtagaacacttggcagatctcgaatctccttggaattgcccacacggacgaccaacaatgcgtcatcttgtggacttgacaactttactcacattacctgatgacgacaatgtcaatgatgatgatgatgatgatgcaaccatctcattggcatga

SEQ ID NO: 31 - protein, 779 aa, Arabidopsis thaliana
MQGDSSPSPTTTSSPLIRPINRNVIHRICSGQVILDLSSAVKELVENSLDAGATSIEINLRDYGEDYFQVIDNGCGISPTNFKVCVQILRRTFDVLALKHHTSKLEDFTDLLNLTTYGFRGEALSSLCALGNLTVETRTKNEPVATLLTFDHSGLLTAEKKTARQIGTTVTVRKLFSNLPVRSKEFKRNIRKEYGKLVSLLNAYALIAKGVRFVCSNTTGKNPKSVVLNTQGRGSLKDNIITVFGISTFTSLQPGTGRNLADRQYFFINGRPVDMPKVSKLVNELYKDTSSRKYPVTILDFIVPGGACDLNVTPDKRKVFFSDETSVIGSLREGLNEIYSSSNASYIVNRFEENSEQPDKAGVSSFQKKSNLLSEGIVLDVSSKTRLGEAIEKENPSLREVEIDNSSPMEKFKFEIKACGTKKGEGSLSVHDVTHLDKTPSKGLPQLNVTEKVTDASKDLSSRSSFAQSTLNTFVTMGKRKHENISTILSETPVLRNQTSSYRVEKSKFEVRALASRCLVEGDQLDDMVISKEDMTPSERDSELGNRISPGTQADNVERHERVLGQFNLGFIIAKLERDLFIVDQHAADEKFNFEHLARSTVLNQQPLLQPLNLELSPEEEVTVLMHMDIIRENGFLLEENPSAPPGKHFRLRAIPYSKNITFGVEDLKDLISTLGDNHGECSVASSYKTSKTDSICPSRVRAMLASRACRSSVMIGDPLRKNEMQKIVEHLADLESPWNCPHGRPTMRHLVDLTTLLTLPDDDNVNDDDDDDATISLA

SEQ ID NO: 32 - DNA, 3456 bp, Arabidopsis thaliana
atgaagacgatcaagcccttgccggaaggagttcgtcactccatgcgttctggaattatcatgttcgacatggcgagggtcgtggaagaactcgtcttcaacagtctcgatgctggggcgaccaaggtgtctatcttcgtgggtgttgtttcatgctctgtgaaagttgtggatgatggatcaggcgtttcaagagatgatttggttttgttgggagaaagatatgctacttcaaagtttcacgacttcaccaacgtggagacagctagtgaaacttttggatttcgtggagaggccttagcttcaatatcagatatctcgttactggaggttaggacaaaagctattgggaggcctaatggttatcgaaaggttatgaagggatccaagtgtctacatcttggaattgatgatgatagaaaagactctggcacgacggtaactgtccgagatctattttacagtcagccagtgagacgaaaatatatgcaatccagccccaagaaagttttggaatctatcaaaaagtgtgtgttccggattgcccttgtgcactccaatgtttccttcagtgttcttgatatcgaaagtgatgaagagcttttccaaaccaatccttcttcttcagcattctcactactgatgagagatgcagggaccgaagctgtaaattcgctttgtaaagtaaacgttacagatggcatgctgaatgtctctggttttgagtgtgcggatgactggaagcctacggatgggcaacaaacaggaagacgcaatagacttcaatccaaccctggttacattctgtgcatagcatgtccacgccgtctttatgaattctcgtttgaaccatcaaagacgcacgttgagttcaagaagtggggacctgtacttgcctttatagaaagaatcactctagccaactggaagaaagatagaattcttgaactttttgatgggggagctgatatactggcaaaaggtgatagacaagacctgattgatgacaaaattagacttcaaaacggcagccttttctcaattcttcattttctggatgcagattggccagaagctatggaacctgcaaaaaagaagctgaagagaagtaatgatcatgcaccttgtagttctctcttgtttccgtctgctgactttaaacaagatggtgattatttttctccacgaaaggatgtatggtctccagaatgtgaagtcgaactgaaaattcagaatcccaaagagcaaggtactgtagctggatttgaaagccggactgattctcttctacagtcacgtgacatagaaatgcaaacgaatgaagacttcccacaagttactgacctccttgaaacaagcttggttgctgactctaagtgccgtaaacagtttctaacaagatgtcagattaccacacctgtcaatatcaaccatgattttatgaaagattcagacgtgttaaattttcagtttcaaggattgaaagatgagttggatgtcagcaattgcattggaaagcatctcttgcgtggttgctcttcaagagtaagcctaacctttcatgagcctaaactatctcatgttgaagggtatgaatccgtcgtgcctatgatacctaatgaaaaacaaagtagtccgcgggtcctagagaccagagaaggtggttcgtactgtgatgtttattctgataagactcctgattgttccctagggagttcatggcaggatactgattggtttactccacagtgttcctcagataggggatgtgttggaattggagaagattttaacattacccccatagatactgcggaatttgattcttatgatgaaaaagttggtagtaaaaagtatctttcttctgtcaatgtggggagctctgttactggtagtttctgtttaagttctgagtggtctccaatgtactccacaccttctgcgaccaagtgggagtctgagtaccagaaaggttgtcgaattcttgaacagagtttgagactgggaaggatgcctgaccctgaattttgtttcagtgcagctaacaacatcaaatttgaccacgaggtcatacctgaaatggattgctgtgaaaccggtacagactctttcacagctattcagaactgcactcagttagctgataaaatttgcaagtcttcgtgggggcatgcagatgatgtgcgtattgaccaatatagtatcaggaaggaaaagttcagttatatggatggcacacagaacaatgctggtaaacaaaggtcaaaaagaagtcgatctgctcctccattttatcgagagaagaagagatttatcagcttaagttgtaaatcagacacaaaaccaaagaactctgatccatcagaacctgatgatctggagtgtttgacacaaccttgtaatgcatctcaaatgcatcttaagtgcagcatccttgatgatgtgtcgtatgaccacatacaagaaacagaaaaaagattgagttctgcctcagacttgaaagcatctgctggttgcaggactgtgcactcagagacccaagatgaggatgtgcacgaagacttcagctcagaggaatttctggatccaattaaatccacaacaaaatggcgccataactgtgcggtctctcaggttcccaaggaatcacacgagcttcatggtcaagatggtgtatttgatatatcttcgggacttctgcacttacgatccgatgaatccttggttcctgaatctatcaacagacactcccttgaagatgccaaggttctacaacaggttgataaaaaatatatcccaatcgttgcttgtggaacagttgccatcgttgatcagcatgctgccgatgaaagaattcgtttggaagagctgcgtacaaagtttattaatgatgcattattaatttttgtgttgacattaaaggtactgccggagatgggttatcagttactccagagttattcagagcagataagagactggggttggatctgcaacattactgtagaagggtcaacgtcctttaagaaaaacatgagcatcatccagcggaaaccaacaccaatcacacttaatgcggttccatgcattctgggtgtaaatctatcagatgttgatctattagagtttcttcagcagcttgctgatactgacggatcatcaactattcctccatctgttcttcgagtcctaaattccaaagcctgtagaggtgcaattatgtttggagatagtctgttaccgtcagaatgctctttaatcattgatggactgaagcagacctcactttgtttccagtgtgctcatgggcgacctacaacagttcctcttgtcgatttgaaggcattgcacaaacagatagcaaagctcagtggaagacaagtgtggcatggcttacaacgcagagaaattacacttgatcgtgcaaaatcacgcttagacaacgctaaaagttaa

SEQ ID NO: 33 - protein, 1151 aa, Arabidopsis thaliana
MKTIKPLPEGVRHSMRSGIIMFDMARVVEELVFNSLDAGATKVSIFVGVVSCSVKVVDDGSGVSRDDLVLLGERYATSKFHDFTNVETASETFGFRGEALASISDISLLEVRTKAIGRPNGYRKVMKGSKCLHLGIDDDRKDSGTTVTVRDLFYSQPVRRKYMQSSPKKVLESIKKCVFRIALVHSNVSFSVLDIESDEELFQTNPSSSAFSLLMRDAGTEAVNSLCKVNVTDGMLNVSGFECADDWKPTDGQQTGRRNRLQSNPGYILCIACPRRLYEFSFEPSKTHVEFKKWGPVLAFIERITLANWKKDRILELFDGGADILAKGDRQDLIDDKIRLQNGSLFSILHFLDADWPEAMEPAKKKLKRSNDHAPCSSLLFPSADFKQDGDYFSPRKDVWSPECEVELKIQNPKEQGTVAGFESRTDSLLQSRDIEMQTNEDFPQVTDLLETSLVADSKCRKQFLTRCQITTPVNINHDFMKDSDVLNFQFQGLKDELDVSNCIGKHLLRGCSSRVSLTFHEPKLSHVEGYESVVPMIPNEKQSSPRVLETREGGSYCDVYSDKTPDCSLGSSWQDTDWFTPQCSSDRGCVGIGEDFNITPIDTAEFDSYDEKVGSKKYLSSVNVGSSVTGSFCLSSEWSPMYSTPSATKWESEYQKGCRILEQSLRLGRMPDPEFCFSAANNIKFDHEVIPEMDCCETGTDSFTAIQNCTQLADKICKSSWGHADDVRIDQYSIRKEKFSYMDGTQNNAGKQRSKRSRSAPPFYREKKRFISLSCKSDTKPKNSDPSEPDDLECLTQPCNASQMHLKCSILDDVSYDHIQETEKRLSSASDLKASAGCRTVHSETQDEDVHEDFSSEEFLDPIKSTTKWRHNCAVSQVPKESHELHGQDGVFDISSGLLHLRSDESLVPESINRHSLEDAKVLQQVDKKYIPIVACGTVAIVDQHAADERIRLEELRTKFINDALLIFVLTLKVLPEMGYQLLQSYSEQIRDWGWICNITVEGSTSFKKNMSIIQRKPTPITLNAVPCILGVNLSDVDLLEFLQQLADTDGSSTIPPSVLRVLNSKACRGAIMFGDSLLPSECSLIIDGLKQTSLCFQCAHGRPTTVPLVDLKALHKQIAKLSGRQVWHGLQRREITLDRAKSRLDNAKS

SEQ ID NO: 34 - DNA, 3330 bp, Arabidopsis thaliana
atgcagcgccagagatcgattttgtctttcttccaaaaacccacggcggcgactacgaagggtttggtttccggcgatgctgctagcggcgggggcggcagcggaggaccacgatttaatgtgaaggaaggggatgctaaaggcgacgcttctgtacgttttgctgtttcgaaatctgtcgatgaggttagaggaacggatactccaccggagaaggttccgcgtcgtgtcctgccgtctggatttaagccggctgaatccgccggtgatgcttcgtccctgttctccaatattatgcataagtttgtaaaagtcgatgatcgagattgttctggagagaggagccgagaagatgttgttccgctgaatgattcatctctatgtatgaaggctaatgatgttattcctcaatttcgttccaataatggtaaaactcaagaaagaaaccatgcttttagtttcagtgggagagctgaacttagatcagtagaagatataggagtagatggcgatgttcctggtccagaaacaccagggatgcgtccacgtgcttctcgcttgaagcgagttctggaggatgaaatgacttttaaggaggataaggttcctgtattggactctaacaaaaggctgaaaatgctccaggatccggtttgtggagagaagaaagaagtaaacgaaggaaccaaatttgaatggcttgagtcttctcgaatcagggatgccaatagaagacgtcctgatgatcccctttacgatagaaagaccttacacataccacctgatgttttcaagaaaatgtctgcatcacaaaagcaatattggagtgttaagagtgaatatatggacattgtgcttttctttaaagtggggaaattttatgagctgtatgagctagatgcggaattaggtcacaaggagcttgactggaagatgaccatgagtggtgtgggaaaatgcagacaggttggtatctctgaaagtgggatagatgaggcagtgcaaaagctattagctcgtggatataaagttggacgaatcgagcagctagaaacatctgaccaagcaaaagccagaggtgctaatactataattccaaggaagctagttcaggtattaactccatcaacagcaagcgagggaaacatcgggcctgatgccgtccatcttcttgctataaaagagatcaaaatggagctacaaaagtgttcaactgtgtatggatttgcttttgttgactgtgctgccttgaggttttgggttgggtccatcagcgatgatgcatcatgtgctgctcttggagcgttattgatgcaggtttctccaaaggaagtgttatatgacagtaaagggctatcaagagaagcacaaaaggctctaaggaaatatacgttgacagggtctacggcggtacagttggctccagtaccacaagtaatgggggatacagatgctgctggagttagaaatataatagaatctaacggatactttaaaggttcttctgaatcatggaactgtgctgttgatggtctaaatgaatgtgatgttgcccttagtgctcttggagagctaattaatcatctgtctaggctaaagctagaagatgtacttaagcatggggatatttttccataccaagtttacaggggttgtctcagaattgatggccagacgatggtaaatcttgagatatttaacaatagctgtgatggtggtccttcagggaccttgtacaaatatcttgataactgtgttagtccaactggtaagcgactcttaaggaattggatctgccatccactcaaagatgtagaaagcatcaataaacggcttgatgtagttgaagaattcacggcaaactcagaaagtatgcaaatcactggccagtatctccacaaacttccagacttagaaagactgctcggacgcatcaagtctagcgttcgatcatcagcctctgtgttgcctgctcttctggggaaaaaagtgctgaaacaacgagttaaagcatttgggcaaattgtgaaagggttcagaagtggaattgatctgttgttggctctacagaaggaatcaaatatgatgagtttgctttataaactctgtaaacttcctatattagtaggaaaaagcgggctagagttatttctttctcaattcgaagcagccatagatagcgactttccaaattatcagaaccaagatgtgacagatgaaaacgctgaaactctcacaatacttatcgaactttttatcgaaagagcaactcaatggtctgaggtcattcacaccataagctgcctagatgtcctgagatcttttgcaatcgcagcaagtctctctgctggaagcatggccaggcctgttatttttcccgaatcagaagctacagatcagaatcagaaaacaaaagggccaatacttaaaatccaaggactatggcatccatttgcagttgcagccgatggtcaattgcctgttccgaatgatatactccttggcgaggctagaagaagcagtggcagcattcatcctcggtcattgttactgacgggaccaaacatgggcggaaaatcaactcttcttcgtgcaacatgtctggccgttatctttgcccaacttggctgctacgtgccgtgtgagtcttgcgaaatctccctcgtggatactatcttcacaaggcttggcgcatctgatagaatcatgacaggagagagtacctttttggtagaatgcactgagacagcgtcagttcttcagaatgcaactcaggattcactagtaatccttgacgaactgggcagaggaactagtactttcgatggatacgccattgcatactcggtttttcgtcacctggtagagaaagttcaatgtcggatgctctttgcaacacattaccaccctctcaccaaggaattcgcgtctcacccacgtgtcacctcgaaacacatggcttgcgcattcaaatcaagatctgattatcaaccacgtggttgtgatcaagacctagtgttcttgtaccgtttaaccgagggagcttgtcctgagagctacggacttcaagtggcactcatggctggaataccaaaccaagtggttgaaacagcatcaggtgctgctcaagccatgaagagatcaattggggaaaacttcaagtcaagtgagctaagatctgagttctcaagtctgcatgaagactggctcaagtcattggtgggtatttctcgagtcgcccacaacaatgcccccattggcgaagatgactacgacactttgttttgcttatggcatgagatcaaatcctcttactgtgttcccaaataa

SEQ ID NO: 35 - protein, 1109 aa, Arabidopsis thaliana
MQRQRSILSFFQKPTAATTKGLVSGDAASGGGGSGGPRFNVKEGDAKGDASVRFAVSKSVDEVRGTDTPPEKVPRRVLPSGFKPAESAGDASSLFSNIMHKFVKVDDRDCSGERSREDVVPLNDSSLCMKANDVIPQFRSNNGKTQERNHAFSFSGRAELRSVEDIGVDGDVPGPETPGMRPRASRLKRVLEDEMTFKEDKVPVLDSNKRLKMLQDPVCGEKKEVNEGTKFEWLESSRIRDANRRRPDDPLYDRKTLHIPPDVFKKMSASQKQYWSVKSEYMDIVLFFKVGKFYELYELDAELGHKELDWKMTMSGVGKCRQVGISESGIDEAVQKLLARGYKVGRIEQLETSDQAKARGANTIIPRKLVQVLTPSTASEGNIGPDAVHLLAIKEIKMELQKCSTVYGFAFVDCAALRFWVGSISDDASCAALGALLMQVSPKEVLYDSKGLSREAQKALRKYTLTGSTAVQLAPVPQVMGDTDAAGVRNIIESNGYFKGSSESWNCAVDGLNECDVALSALGELINHLSRLKLEDVLKHGDIFPYQVYRGCLRIDGQTMVNLEIFNNSCDGGPSGTLYKYLDNCVSPTGKRLLRNWICHPLKDVESINKRLDVVEEFTANSESMQITGQYLHKLPDLERLLGRIKSSVRSSASVLPALLGKKVLKQRVKAFGQIVKGFRSGIDLLLALQKESNMMSLLYKLCKLPILVGKSGLELFLSQFEAAIDSDFPNYQNQDVTDENAETLTILIELFIERATQWSEVIHTISCLDVLRSFAIAASLSAGSMARPVIFPESEATDQNQKTKGPILKIQGLWHPFAVAADGQLPVPNDILLGEARRSSGSIHPRSLLLTGPNMGGKSTLLRATCLAVIFAQLGCYVPCESCEISLVDTIFTRLGASDRIMTGESTFLVECTETASVLQNATQDSLVILDELGRGTSTFDGYAIAYSVFRHLVEKVQCRMLFATHYHPLTKEFASHPRVTSKHMACAFKSRSDYQPRGCDQDLVFLYRLTEGACPESYGLQVALMAGIPNQVVETASGAAQAMKRSIGENFKSSELRSEFSSLHEDWLKSLVGISRVAHNNAPIGEDDYDTLFCLWHEIKSSYCVPK

SEQ ID NO: 36 - DNA, 1170 bp, Homo sapiens
atggcgcaaccaaagcaagagagggtggcgcgtgccagacaccaacggtcggaaaccgccagacaccaacggtcggaaaccgccaagacaccaacgctcggaaaccgccagacaccaacgctcggaaaccgccagacaccaaggctcggaatccacgccaggccacgacggagggcgactacctcccttctgaccctgctgctggcgttcggaaaaaacgcagtccggtgtgctctgattggtccaggctctttgacgtcacggactcgacctttgacagagccactaggcgaaaaggagagacgggaagtattttttccgccccgcccggaaagggtggagcacaacgtcgaaagcagccgttgggagcccaggaggcggggcgcctgtgggagccgtggagggaactttcccagtccccgaggcggatccggtgttgcatccttggagcgagctgagaactcgagtacagaacctgctaaggccatcaaacctattgatcggaagtcagtccatcagatttgctctgggccggtggtaccgagtctaaggccgaatgcggtgaaggagttagtagaaaacagtctggatgctggtgccactaatgttgatctaaagcttaaggactatggagtggatctcattgaagtttcaggcaatggatgtggggtagaagaagaaaacttcgaaggctttactctgaaacatcacacatgtaagattcaagagtttgccgacctaactcaggtggaaacttttggctttcggggggaagctctgagctcactttgtgcactgagtgatgtcaccatttctacctgccgtgtatcagcgaaggttgggactcgactggtgtttgatcactatgggaaaatcatccagaaaaccccctacccccgccccagagggatgacagtcagcgtgaagcagttattttctacgctacctgtgcaccataaagaatttcaaaggaatattaagaagaaacgtgcctgcttccccttcgccttctgccgtgattgtcagtttcctgaggcctccccagccatgcttcctgtacagcctgtagaactgactcctagaagtaccccaccccacccctgctccttggaggacaacgtgatcactgtattcagctctgtcaagaatggtccaggttcttctagatga

SEQ ID NO: 37 - protein, 389 aa, Homo sapiens; identical to SEQ ID NO: 18

SEQ ID NO: 38 - DNA, 795 bp, Homo sapiens; identical to SEQ ID NO: 21

SEQ ID NO: 39 - protein, 264 aa, Homo sapiens; identical to SEQ ID NO: 20

(19) United States
(12) Patent Application Publication
(10) Pub. No.: US 2003/0068808 A1
(43) Pub. Date: Apr. 10, 2003

(54) Methods for generating antibiotic resistant microbes and novel antibiotics

(76) Inventors: Nicholas C. Nicolaides, Boothwyn, PA (US); Philip M. Sass, Audubon, PA (US); Luigi Grasso, Philadelphia, PA (US); J. Bradford Kline, Blue Bell, PA (US)

Correspondence Address: Woodcock Washburn Kurtz, Mackiewicz & Norris LLP, 46th Floor, One Liberty Place, Philadelphia, PA, 19103, US

(21) Appl. No.: 09/912,697

(22) Filed: Jul. 25, 2001

(51) Int. Cl. C12N 15/01; C12N 1/21

(52) U.S. Cl. 435/252.3; 435/443

(57) ABSTRACT

The invention provides methods for generating antibiotic resistant bacteria comprising blocking mismatch repair in a bacterium to make hypermutable bacteria, contacting the bacteria with at least one antibiotic, selecting bacteria that are resistant to the antibiotic, and culturing the antibiotic resistant bacteria. The invention also provides methods of determining the genes responsible for antibiotic resistance.

### FIELD OF THE INVENTION

This invention relates to the field of antimicrobial treatments and gene targets for the discovery of antimicrobial agents. In particular, it relates to the discovery of genes essential for growth and virulence of bacteria.

Despite the development of new classes of antimicrobial agents over the past decade (reviewed in http://vet.purdue.edu/bms), microbial infections remain a serious health problem. While antibiotics treatment has been effective in controlling infectious diseases, an increase in the number of antibiotic-resistant (AR) microbes have emerged and are now posing a major therapeutic problem. In today's industrialized societies, infectious strains can be found that are resistant to all classes of antimicrobial agents used in the clinic. Infections due to resistant strains include higher morbidity and mortality, longer patient hospitalization, and an increase in treatment costs (Murray (1994) New Engl. J. Med. 330:1229-1230). In light of these findings, an unmet need exists for the development of new therapeutic agents that can work by inhibiting the ever-increasing number of novel antibiotic resistance mechanisms.

One approach for generating new therapies and/or therapeutic strategies against AR microbes is to develop methods that can generate a wide array of genomic alterations in a microbe's genome that can yield maximal number altered target genes that are capable of eliciting antibiotic resistance. Once an AR strain is developed, it can be used for rapid genome analysis to identify mutant gene(s) that are capable of rendering a microbe resistant to an antibiotic for target identification. Such analysis can involve any of a variety of methods used by those skilled in the art for identifying mutations and/or differential gene expression, including but not limited to differential gene expression using microarrays, cDNA subtraction, differential protein analysis, complementation assays, single nucleotide polymorphosm (SNP) analysis or whole genome sequencing to identify altered loci.

A bottleneck to generating genetically diverse microbes is the inability to generate nonbiased genome-wide mutations. Many mutagenesis methods are available whereby the use of chemical and radiation exposure has been successful in generating genomic mutations. A limitation of this approach is that these various methods are usually DNA site specific or are extremely toxic, therefore limiting the mutation spectra and the opportunity to identify a maximal number of genes, when mutated, that are able to confer resistance to an antibiotic. Recently, work done by Nicolaides, et al. (Nicolaides et al. (1998) Mol. Cell. Biol. 18:1635-1641; U.S. Pat. No. 6,146,894) has demonstrated the utility of introducing dominant negative mismatch repair mutants into cells to confer global DNA hypermutability. These mutations are in the form of point mutations or small insertion-deletions that are distributed equally throughout the genome. The ability to manipulate the mismatch repair (MMR) process of a target host organism can lead to an increase in the mutability of the target host genome, leading to the generation of innovative cell subtypes with varying phenotypes from the original wild-type cells. Variants can be placed under a specified, desired selective process the result of which is the capacity to select for a novel organism that expresses an altered biological molecule(s) and has a new phenotype. The concept of creating and introducing dominant negative allele of a gene, including the MMR alleles, in bacterial cells has been documented to result in genetically altered prokaryotic mismatch repair genes (Aronshtam and Marinus (1996) Nucl. Acids Res. 24:2498-2504; Wu and Marinus (1994) J. Bacteriol. 176:5393-400; Brosh and Matson (1995) J. Bacteriol. 177:5612-5621). Furthermore, altered MMR activity has been demonstrated when MMR genes from different species including yeast and mammalian cells are over-expressed (Fishel et al. (1993) Cell 7:1027-1038; Lipkin et al. (2000) Nat. Genet. 24:27-35). The ability to create hypermutable organisms by blocking MMR has great commercial value for the generation of AR bacterial strains for drug screening and target discovery.

There is an urgent need in the art to elucidate the mechanisms of antimicrobial resistance, and to identify novel antimicrobial agents.

### SUMMARY

The invention provides new uses of MMR deficiency in bacteria to identify antibiotic resistance (AR) genes and pathways that can lead to the generation of novel therapeutic strategies for alternative clinical strategies.

Antibiotic resistant (AR) microbes express a number of genes that are essential for growth of the organism in an infection, and serve as useful reagents for target discovery and/or screening lines for the discovery of novel antimicrobial agents. This invention provides an approach to the identification of genes that confer anti-microbial resistance, and the use of those genes, and bacterial strains expressing mutant forms of genes, in the identification, characterization, and evaluation of targets for therapeutic development. In addition, this application teaches of the use of employing structural information of the gene, gene product and mutant strains in screening for antimicrobial agents active against the genes and their corresponding products and pathways. Positive compounds can then be used as final products or precursors to be further developed into antibacterial agents. This invention also provides methods of treating microbial infections in mammals by administering an antimicrobial agent active against such an identified target gene or product, and the pharmaceutical compositions effective for such treatment.

To identify genes capable of rendering bacteria antibiotic-resistant, the invention provides methods of decreasing MMR activity of a microbial host to produce AR strains. Using this process, commercially viable microbes that are resistant to a wide range of antibiotics can be directly selected for the resistance to an anti-microbial agent of interest. AR microbes may be genetically screened to identify novel therapeutic targets for drug development. Once a bacterium with a specified resistance is isolated, the MMR activity may be restored by several methods well known to those skilled in the art as a means to gentically “fix” the new mutations in the host genome, thereby making the AR microbe suitable for comparative molecular analysis to the wild-type strain as well as for drug screening to identify novel antimicrobial compounds. For example, if MMR is decreased by the use of a dominant-negative allele or antisense vector directed to an internal MMR gene, the endogenous repair activity can be restored if the gene is expressed by an inducible promoter system, including but not limited to promoters such as: TAC-LACI, tryp (Brosius et al. (1984) Gene 27:161-172), araBAD (Guzman et al. (1995) J. Bact. 177:4121-4130) pLex (La Vallie et al. (1992) Bio. Technology 11:187-193), pRSET (Schoepfer, R. (1993) Gene 124:83-85), pT7 (Studier (1991) J. Mol. Biol. 219(l):37-44) etc., by removing the inducer and, therefore, reducing the the promoter activity. In the case that the expression vector employs a Cre-lox system, MMR can be restored by disrupting the cDNA gene insert from the host cell harboring the expression vector (Hasan, N. et al. (1994) Gene 2:51-56). Yet other methods include homologous knockout of the expression vector that can turn off the actively expressed gene used to inhibit MMR activity. In addition to the recombinant methods outlined above that have the capacity to eliminate the MMR activity from the microbe, it has been demonstrated that many chemicals have the ability to “cure” microbial cells of plasmids. For example, chemical treatment of cells with drugs including bleomycin (Attfield et al. (1985) Antimicrob. Agents Chemother. 27:985-988) or novobiocin, coumercycin, and quinolones (Fu et al. (1988) Chemotherapy 34:415-418) have been shown to result in microbial cells that lack endogenous plasmid as evidenced by Southern analysis of cured cells as well as sensitivity to the appropriate antibiotic (Attfield et al. (1985) Antimicrob. Agents Chemother. 27(6):985-988, Fu et al. (1988) Chem. Abstracts 34(5):415-418; BiWang et al. (1999) J. of Fujian Agricultural University 28(1):43-46; Brosius, J. (1988) Biotechnology 10:205-225). Whether by use of recombinant means or treatment of cells with chemicals, removal of the MMR-expression plasmid results in the reestablishment of a genetically stable microbial cell line. Therefore, the restoration of MMR allows host bacteria to function normally to repair DNA. The newly generated mutant bacterial strain that exhibits a novel anti-microbial resistance is now suitable for gene/protein discovery to identify new biomolecules that are involved in generating resistance as well as a model system to screen for novel anti-microbial agents targeted against certain antibiotic resistant strains.

In certain embodiments, the invention provides methods for generating antibiotic resistant bacteria comprising the steps of:

blocking mismatch repair in the bacterium whereby the bacterium becomes hypermutable;

contacting the bacterium with at least one antibiotic determining whether the bacterium is resistant to the antibiotic, thereby generating antibiotic resistant bacteria.

In the methods of the invention, mismatch repair may be blocked in some embodiments by introducing a polynucleotide encoding a wild-type allele of a mismatch repair gene into a cell, whereby the wild-type allele inactivates the endogenous MMR activity by binding to and interfering with the resident activity. The cell becomes hypermutable as a result of the introduction of the gene.

In other embodiments of the invention, a polynucleotide encoding a dominant negative allele of a mismatch repair gene is introduced into a cell, where the dominant negative gene is derived from a mismatch repair gene from a different organism. The cell becomes hypermutable as a result of the introduction of the gene. In particular embodiments of this method, MMR activity is inhibited for ten rounds of cell division and then the MMR activity is restored therefore restoring the genetic stability. An example of a dominant negative MMR gene is the PMS2-134 gene.

In other embodiments of the invention, a polynucleotide encoding an allele of a mismatch repair gene is introduced into a bacterial cell, where the mismatch repair gene is derived from a wild-type or altered mammalian, yeast, fungal, amphibian, insect, plant or bacterial mismatch repair gene. The cell becomes hypermutable as a result of the introduction of the gene.

In another embodiment, mismatch repair may be blocked by introducing an antisense nucleic acid molecule into the bacterium wherein the antisense nucleic acid molecule specifically binds to a mismatch repair gene and inhibits mismatch repair in the bacterium.

In other embodiments of the invention, methods are provided for generating a genetic alteration of a bacterial host genome to produce variant strains expressing new output traits. Transgenic bacterium comprising a polynucleotide encoding a wild-type allele of a mismatch repair gene is grown. The bacteria are comprised of a set of altered genes for a desired biological phenotype.

In other embodiments of the invention, methods are provided for generating a genetic alteration of a bacterial host genome to produce variant strains that are resistant to antimicrobial agents. Bacteria with decreased mismatch repair are grown. The bacteria are comprised of a set of altered genes for a desired antibiotic-resistance phenotype.

In further embodiments of the invention, methods are provided for creating a hypermutable bacterium using a wild-type MMR allele for antibiotic-resistance selection, and restoring genomic stability of a selected host by inactivating or decreasing the expression of the wild-type MMR allele.

In another embodiment of the invention, a method is provided for creating a hypermutable bacteria using a dominant negative MMR allele for antibiotic-resistance selection, and restoring genomic stability of a selected host by inactivating or decreasing the expression of the dominant negative MMR gene allele.

In another embodiment of the invention, a method is provided for creating a hypermutable bacteria expressing an antisense gene to a MMR gene for antibiotic-resistance selection, and restoring genomic stability of a selected host by inactivating or decreasing the expression of the dominant negative MMR gene allele.

In another embodiment of the invention, a method is provided for creating a hypermutable bacteria using chemical inhibitors of MMR for antibiotic-resistance selection, and restoring genomic stability of a selected host by removing the chemical inhibitor by introducing a dominant negative allele of a mismatch repair gene into the bacterium. The dominant negative allele may be, for example, a PMS2-134 gene.

In another embodiment, mismatch repair may be blocked by exposing the bacterium a to a compound that inhibits mismatch repair whereby cells are grown in the presence of the compound and undergo multiple rounds of cell divison in the absence of MMR, yielding sibs that are genetically diverse. Sibs are then selected for antibiotic resistance. AR strains are removed from chemical inhibitor and the endogenous MMR activity is restored leaving genetically stable strains that are now suitable for gene discovery and/or therapeutic agent development. For example, the compound that blocks mismatch repair may be an anthracene derivative, including, but not limited 1,2-dimethylanthracene, 9,10-dimethyl anthracene, 7,8-dimethylanthracene, 9,10-diphenylanthracene, 9,10-dihydroxymethylanthracene, 9-hydroxymethyl-10-methylanthracene, dimethylanthracene-1,2-diol, 9-hydroxymethyl-10-methylanthracene-1,2-diol, 9-hydroxymethyl-10-methylanthracene-3,4-diol, 9, 10-di-m-tolyanthracene. In other embodiments, the compound that blocks MMR activity is an ATP analog. In other embodiments, the compound that blocks MMR activity is a nuclease inhibitor. In other embodiments, the compound that blocks MMR activity is a DNA polymerase inhibitor.

The methods of the invention may further comprise exposing the bacteria to chemical mutagens. While it has been documented that MMR deficiency can lead to as much as a 1000-fold increase in the endogenous DNA mutation rate of a host, there is no assurance that MMR deficiency alone will be sufficient to alter every gene within the DNA of the host bacterium to create altered biochemicals with new activity(s). Therefore, the use of chemical agents and their respective analogues such as methane sulfonate, dimethyl sulfonate, O-6-methyl benzadine, ethylnitrosourea (ENU), ethidium bromide, ethyl methanesulfonate (EMS), N-methyl-N′-nitro-N-nitrosoguanidine (MNNG), methylnitrosourea (MNU), Tamoxifen, 8-hydroxyguanine, as well as others listed but not limited to in publications by: Khromov-Borisov et al (1999) Mutat. Res. 430:55-74; Ohe et al. (1999) Mutat. Res. 429:189-199; Hour et al. (1999) Food Chem. Toxicol. 37:569-579; Hrelia et al. (1999) Chem. Biol. Interact. 118:99-111; Garganta et al. (1999) Environ. Mol. Mutagen. 33:75-85; Ukawa-Ishikawa et al (1998) Mutat. Res. 412:99-107; www.ehs.utah.edu/ohh/mutagens, etc. can be used in the methods of the invention to further enhance the spectrum of mutations and increase the likelihood of obtaining alterations in one or more genes that can in turn generate host bacteria with a complex antibiotic resistant phenotype (Fu et al. (1988) Chemotherapy 34(5):415-418; Lee et al. (1994) Mutagenesis 9:401-405; Vidal et al. (1995) Carcinogenesis 16:817-821). Prior art teaches us that mismatch repair deficiency leads to hosts with an increased resistance to toxicity by chemicals with DNA damaging activity. This feature allows for the creation of additional genetically diverse hosts when MMR defective bacteria are exposed to such agents, which would be otherwise impossible due to the toxic effects of such chemical mutagens (Colella et al. (1999) Br. J. Cancer 80:338-343; Moreland et al. (1999) Cancer Res. 59:2102-2106; Humbert et al. (1999) Carcinogenesis 20:205-214; Glaab et al. (1998) Mutat. Res. 398:197-207). Moreover, prior art teaches us that MMR is responsible for repairing chemical-induced DNA adducts, so therefore blocking this process could theoretically increase the number, types, mutation rate and genomic alterations of a bacterial host [Rasmussen et al. (1996) Carcinogenesis 17:2085-2088; Sledziewska-Gojska et al. (1997) Mutat. Res. 383:31-37; Janion et al. (1989) Mutat. Res. 210:15-22). In addition to the chemicals listed above, other types of DNA mutagens include ionizing radiation and UV-irradiation, which are known to cause DNA mutagenesis in bacteria can also be used to potentially enhance this process. These agents, which are extremely toxic to host cells and, therefore, result in a decrease in the actual pool size of altered bacterial cells, are more tolerated in MMR defective hosts and in turn allow for a enriched spectrum and degree of genomic mutation ( Drummond et al. (1996) J. Biol. Chem. 271:9645-19648). such as, but not limited to methane sulfonate, dimethyl sulfonate, O-6-methyl benzadine, ethylnitrosourea, ethidium bromide, ethyl methanesulfonate, N-methyl-N′-nitro-N-nitrosoguanidine, methylnitrosourea, Tamoxifen, and 8-hydroxyguanine.

The methods of the invention may be used to generate AR bacteria which are resistant to such antibiotic compounds as, for example, quinilones, aminoglycosides, magainins, defensins, tetracyclines, beta-lactams, macrolides, lincosamide, sulfonamides, chloramphenicols, nitrofurantoins, and isoniazids.

In the methods of the invention, the step of determining whether the bacterium is resistant to an antibiotic may comprise analyzing the bacterium for multiantiboitic resistance.

Further, the methods of the invention may comprise making antibiotic resistant bacteria genetically stable, such as by removing the MMR inhibitory molecule, for example.

In the methods of the invention, the genome of the antibiotic resistant bacterium and the genome of a wild-type strain of the bacterium may be compared by sequence analysis of the entire genomes, or compared by microarray analysis, for example.

In another embodiment, the genome of said antibiotic resistant bacterium and the genome of said wild-type strain of said bacterium are compared by:

introducing gene fragments from the antibiotic resistant bacterium into the wild-type bacterium, thereby producing mutant bacteria;

selecting a mutant bacterium with antibiotic resistance; and sequencing the gene fragment from the mutant bacterium with antibiotic resistance, thereby identifying the antibiotic resistant gene.

The invention also provides methods of using microbial strains that are naturally defective for MMR due to defects in genes encoding for MMR proteins. Strains in which muts, mutL, muth, or mutY genes are defective have been reported to be defective in MMR activity (Modrich (1994) Science 266:1959-1960). The methods of the invention may employ bacterial strains with mutant endogenous MMR genes for selecting for variants that are AR. Once an AR variant strain is identified, the genetic stability of the microbe can be restored by expressing a functional gene that can complement the defective MMR gene activity.

Mutant strains can be used for gene identification by isolating DNA fragments derived from the MMR defective antibiotic-resistant strains. These bacteria contain DNA fragments with altered sequences that can be introduced into wild-type counterparts (antibiotic susceptible) and screened for fragments that confer antibiotic resistance. Conversely, DNA fragments derived from the wild-type bacteria can be introduced into mutant bacterial strains to screen for genes effective via loss-of-function mutated genes. The fact that a clone is complemented suggests the introduced fragment contains a gene encoding for an antibiotic-resistant gene product. Other methods can also be used to identify AR genes including but not limited to microarray analysis of gene expression, differential expression and/or differential protein analysis know by those skilled in the art.

The microbial strains described herein have either been generated and characterized in a manner which essentially provides a process by which the manipulation of MMR can confer AR against a wide range of anti-microbial compounds and that these strains are now useful for target discovery and/or therapeutic agent discovery as screening lines.

In other embodiments of the invention, methods of producing a stable bacterium expressing a new phenotype is provided. Turning off the expression of the MMR-wild-type alleles, MMR-dominant negative alleles, or MMR-antisense alleles, results in genetically stable bacteria expressing a new output trait(s).

The invention also provides antibiotic resistant strains of bacteria produced by the methods of the invention.

These and other aspects of the invention provide the art with methods that can generate enhanced mutability in bacteria as well as providing prokaryotic organisms harboring potentially useful mutations to generate novel output traits for commercial applications, and are set forth in greater detail below.

### BRIEF DESCRIPTION OF THE FIGURE

FIG. 1 shows growth of tetracyclin-resistant mutant bacteria carrying a dominant negative allele of PMS2 in the pT7Ea plasmid (134/V5), tetracyclin-resistant mutant bacteria carrying a the PMSR3 gene in the pT7Ea plasmid (R3), and wild-type bacteria carrying the empty pT7Ea plasmid (T7), on medium containing tetracyclin at 0, 4 and 6 hours after tetracycline addition.

### DETAILED DESCRIPTION OF THE INVENTION

The inventors present a method for developing hypermutable bacteria by altering the activity of endogenous mismatch repair (MMR) activity of hosts to generate antibiotic resistant (AR) microbes for target discovery and the development of novel anti-microbial agent by screening for new compounds. Wild-type and some dominant negative alleles of mismatch repair genes, when introduced and expressed in bacteria, increase the rate of spontaneous mutations by reducing the effectiveness of the endogenous MMR-mediated DNA repair activity, thereby rendering the bacteria highly susceptible to genetic alterations due to hypermutability. Hypermutable bacteria can then be utilized to screen for novel mutations in a gene or a set of genes that produce variant siblings exhibiting new output traits not found in the wild-type cells such as antibiotic resistance.

The process of mismatch repair, also called mismatch proofreading, is an evolutionarily highly conserved process that is carried out by protein complexes described in cells as disparate as prokaryotic cells such as bacteria to more complex mammalian cells (Modrich (1994) Science 266:1959-1960; Strand et al. (1993) Nature 365:274-276; Su et al. (1988) J. Biol. Chem. 263:6829-6835; Aronshtam.and Marinus (1996) Nucl. Acids Res. 24:2498-2504; Wu and Marinus (1994) J. Bacteriol. 176:5393-400). A mismatch repair gene is a gene that encodes one of the proteins of such a mismatch repair complex. Although not wanting to be bound by any particular theory of mechanism of action, a mismatch repair complex is believed to detect distortions of the DNA helix resulting from non-complementary pairing of nucleotide bases. The non-complementary base on the newer DNA strand is excised, and the excised base is replaced with the appropriate base that is complementary to the older DNA strand. In this way, cells eliminate many mutations that occur as a result of mistakes in DNA replication, resulting in genetic stability of the sibling cells derived from the parental cell.

Some wild-type MMR gene alleles as well as dominant negative alleles cause a mismatch repair defective phenotype even in the presence of a wild-type MMR gene allele in the same cell. An example of a dominant negative allele of a MMR gene is the human gene hPMS2-134, which carries a truncation mutation at codon 134 (Nicolaides et al. (1998) Mol. Cell. Biol. 18:1635-1641). The mutation causes the product of this gene to abnormally terminate at the position of the 134th amino acid, resulting in a shortened polypeptide containing the N-terminal 133 amino acids. Such a mutation causes an increase in the rate of mutations, which accumulate in cells after DNA replication. Expression of a dominant negative allele of a mismatch repair gene results in impairment of mismatch repair activity, even in the presence of the wild-type allele. Any mismatch repair allele, which produces such effect, can be used in this invention. In addition, the use of over-expressed wild-type MMR gene alleles from human, mouse, plants, and yeast in bacteria has been shown to cause a dominant negative effect on the bacterial hosts MMR activity (Fishel et al. (1993) Cell 7:1027-1038; Aronshtam and Marinus (1996) Nucl. Acids Res. 24:2498-2504; Wu and Marinus (1994) J. Bacteriol. 176:5393-400; Lipkin et al. (2000) Nat. Genet. 24:27-35).

Dominant negative alleles of a mismatch repair gene can be obtained from the cells of humans, animals, yeast, bacteria, plants or other organisms. Screening cells for defective mismatch repair activity can identify such alleles. Mismatch repair genes may be mutant or wild-type. Bacterial host MMR may be mutated or not. The term bacteria used in this application include any organism from the prokaryotic kingdom. These organisms include genera such as but not limited to Agrobacterium, Anaerobacter, Aquabacterium, Azorhizobium, Bacillus, Bradyrhizobium, Cryobacterium, Escherichia, Enterococcus, Heliobacterium, Klebsiella, Lactobacillus, Methanococcus, Methanothermobacter, Micrococcus, Mycobacterium, Oceanomonas, Pseudomonas, Rhizobium, Staphylococcus, Streptococcus, Streptomyces, Thermusaquaticus, Thermaerobacter, Thermobacillus, etc. Other procaryotes that can be used for this application are listed at (www.bacterio.cict.fr/validgenericnames). Bacteria exposed to chemical mutagens or radiation exposure can be screened for defective mismatch repair. Genomic DNA, cDNA, or mRNA from any cell encoding a mismatch repair protein can be analyzed for variations from the wild-type sequence. Dominant negative alleles of a mismatch repair gene can also be created artificially, for example, by producing variants of the hPMS2-134 allele or other mismatch repair genes (Nicolaides et al. (1998) Mol. Cell. Biol. 18:1635-1641). Various techniques of site-directed mutagenesis can be used. The suitability of such alleles, whether natural or artificial, for use in generating hypermutable bacteria can be evaluated by testing the mismatch repair activity (using methods described in Nicolaides et al. (1998) Mol. Cell. Biol. 18:1635-1641) caused by the allele in the presence of one or more wild-type alleles, to determine if it is a dominant negative allele.

A bacterium that over-expresses a wild-type mismatch repair allele or a dominant negative allele of a mismatch repair gene will become hypermutable. This means that the spontaneous mutation rate of such bacteria is elevated compared to bacteria without such alleles. The degree of elevation of the spontaneous mutation rate can be at least 2-fold, 5-fold, 10-fold, 20-fold, 50-fold, 100-fold, 200-fold, 500-fold, or 1000-fold that of the normal bacteria as measured as a function of bacterial doubling/minute.

According to one aspect of the invention, a polynucleotide encoding either a wild-type or a dominant negative form of a mismatch repair protein is introduced into bacteria. The gene can be any dominant negative allele encoding a protein which is part of a mismatch repair complex, for example, muts, mutL, mutH, or mutYhomologs of the bacterial, yeast, plant or mammalian genes (Modrich (1994) Science 266:1959-1960; Prolla et al. (1994) Science 264:1091-1093). The dominant negative allele can be naturally occurring or made in the laboratory. The polynucleotide can be in the form of genomic DNA, cDNA, RNA, or a chemically synthesized polynucleotide or polypeptide. The molecule can be introduced into the cell by transfection or other methods well described in the literature.

Transfection is any process whereby a polynucleotide or polypeptide is introduced into a cell. The process of transfection can be carried out in a bacterial culture using a suspension culture. The bacteria can be any type classified under the prokaryotes.

In general, transfection will be carried out using a suspension of cells but other methods can also be employed as long as a sufficient fraction of the treated cells incorporate the polynucleotide or polypeptide so as to allow transfected cells to be grown and utilized. The protein product of the polynucleotide may be transiently or stably expressed in the cell. Techniques for transfection are well known to those skilled in the art. Available techniques to introduce a polynucleotide or polypeptide into a prokaryote include but are not limited to electroporation, transduction, cell fusion, the use of chemically competent cells (e.g., calcium chloride), and packaging of the polynucleotide together with lipid for fusion with the cells of interest. Once a cell has been transformed with the inhibitory mismatch repair gene or protein, the cell can be propagated and manipulated in either liquid culture or on a solid agar matrix, such as a petri dish. If the transfected cell is stable, the gene will be retained and expressed at a consistent level when the promoter is constitutively active, or when in the presence of appropriate inducer molecules when the promoter is inducible, for many cell generations, and a stable, hypermutable bacterial strain results.

An isolated bacterial cell is a clone obtained from a pool of a bacterial culture by chemically selecting out strains using antibiotic selection of an expression vector. If the bacterial cell is derived from a single cell, it is defined as a clone.

Bacterial cultures may be screened for antibiotic resistance against a wide array of antibiotic compounds. For example, but not by way of limitation, bacteria produced by the methods of the invention may be screened for resistance to quinilones, aminoglycosides, magainins, defensins, tetracyclines, beta-lactams, macrolides, lincosamide, sulfonamides, chloramphenicols, nitrofurantoins, and isoniazids. The antibiotics may be incorporated into solid or liquid growth medium, for example.

A polynucleotide encoding an inhibitory form of a mismatch repair protein can be introduced into the genome of a bacterium or propagated on an extra-chromosomal plasmid. Selection of clones harboring the mismatch repair gene expression vector can be accomplished by addition of any of several different antibiotics, including but not limited to ampicillin, kanamycin, chloramphenicol, zeocin, and tetracycline. The microbe can be any species for which suitable techniques are available to produce transgenic microorganisms, such as but not limited to genera including Bacillus, Pseudomonas, Staphylococcus, Escherichia and others. Any method for making transgenic bacteria known in the art can be used. According to one process of producing a transgenic microorganism, the polynucleotide is transfected into the microbe by one of the methods well known to those in the art. Next, the microbial culture is grown under conditions that select for cells in which the polynucleotide encoding the mismatch repair gene is either incorporated into the host genome as a stable entity or propagated on a self-replicating extra-chromosomal plasmid, and the protein encoded by the polynucleotide fragment transcribed and subsequently translated into a functional protein within the cell. Once a transgenic microbe is engineered to harbor the expression construct, it is then propagated to generate and sustain a culture of transgenic microbes indefinitely.

Once a stable, transgenic microorganism has been engineered to express a functional MMR protein, the microbe can be exploited to create novel mutations in one or more target gene(s) of interest harbored within the same microorganism. A gene of interest can be any gene naturally possessed by the bacterium or one introduced into the bacterial host by standard recombinant DNA techniques. The target gene(s) may be known prior to the selection or unknown. One advantage of employing such transgenic microbes to induce mutations in resident or extra-chromosomal genes within the microbe is that it is unnecessary to expose the microorganism to mutagenic insult, whether it be chemical or radiation in nature, to produce a series of random gene alterations in the target gene(s). This is due to the highly efficient nature and the spectrum of naturally occurring mutations that result as a consequence of the altered mismatch repair process. However, it is possible to increase the spectrum and frequency of mutations by the concomitant use of either chemical and/or radiation together with MMR defective cells. The net effect of the combination treatment is the increase in altered gene pool in the genetically altered microbe that result in an increased alteration of an allele(s) that are useful for producing new output traits. Other benefits of using MMR-defective microbes that are taught in this application are genetic screens for the DIRECT selection of variant sub-clones that exhibit new output traits with commercially important applications such as antibiotic resistance, which allows the bypassing of the tedious and time consuming gene identification, isolation and characterization stages.

Mutations can be detected by analyzing the recombinant microbe for alterations in the genotype and/or phenotype post-activation of the decreased mismatch repair activity of the transgenic microorganism. Novel genes that produce altered phenotypes in MMR-defective microbial cells can be discerned by any variety of molecular techniques well known to those in the art. For example, the microbial genome can be isolated and a library of restriction fragments cloned into a plasmid vector. The library can be introduced into a “normal” cell and the cells exhibiting the novel phenotype screened. Transformed cells are then screened for the new phenotype (e.g., antibiotic resistance). A plasmid is isolated from those normal, transformed cells that exhibit the novel phenotype and the inserted gene(s) characterized by DNA sequence analysis.

Alternatively, differential messenger RNA screen can be employed utilizing driver and tester RNA (derived from wild-type and novel mutant respectively) followed by cloning the differential transcripts and characterizing them by standard molecular biology methods well known to those skilled in the art. Furthermore, if the mutant sought is on encoded by an extrachromosmal plasmid, then following co-expression of the dominant negative MMR gene and the gene of interest to be altered and phenotypic selection, the plasmid is isolated from mutant clones and analyzed by DNA sequence analysis by methods well known to those in the art.

In another embodiment, the screening of cells may be performed by microarray analysis. In microarray analysis, microchips containing all or a subset of all expressed bacterial genes may be screened using RNA molecules derived from the wild-type or antibiotic resistant strain whereby RNA derived from one strain is reverse transcribed using FluoroLink Cy3 and the other RNA sample is reverse transcribe-labelled using Cy5 dUTP. Labelled cDNAs from each organism are used to probe the microchip whereby unique message from one source will generate a distinct signal while message expressed from both sources will generate a common fluorescence. Alternatively, microchips containing olignucleotide derived from the wild-type strain can be used to hybridize genomic fragments from the antibiotic resistant strain to identify fragments containing a mutated gene by loss of hybridization.

Phenotypic screening for output traits in MMR-defective mutants can be by biochemical activity and/or a physical phenotype of the altered gene product. A mutant phenotype can also be detected by identifying alterations in electrophoretic mobility, DNA binding in the case of transcription factors, spectroscopic properties such as IR, CD, X-ray crystallography or high field NMR analysis, or other physical or structural characteristics of a protein encoded by a mutant gene. It is also possible to screen for altered novel function of a protein in situ, in isolated form, or in model systems. One can screen for alteration of any property of the microorganism associated with the function of the gene of interest, whether the gene is known prior to the selection or unknown. The aforementioned screening and selection discussion is meant to illustrate the potential means of obtaining novel mutants with commercially valuable output traits.

Plasmid expression vectors that harbor the mismatch repair (MMR) gene inserts can be used in combination with a number of commercially available regulatory sequences to control both the temporal and quantitative biochemical expression level of the dominant negative MMR protein. The regulatory sequences can be comprised of a promoter, enhancer or promoter/enhancer combination and can be inserted either upstream or downstream of the MMR gene to control the expression level. The regulatory promoter sequence can be any of those well known to those in the art, including but not limited to the lac, tetracycline, tryptophan-inducible, phosphate inducible, T7-polymerase-inducible (Studier et al. (1991) J. Mol. Biol. 219(l):37-44), and steroid inducible constructs as well as sequences which can result in the excision of the dominant negative mismatch repair gene such as those of the Cre-Lox system. These types of regulatory systems have been listed in scientific publications and are familiar to those skilled in the art.

Once a microorganism with a novel, desired output trait of interest is created, the activity of the aberrant MMR activity is attenuated or eliminated by any of a variety of methods, including removal of the inducer from the culture medium that is responsible for promoter activation, gene disruption of the aberrant MMR gene constructs, electroporation and or chemical curing of the expression plasmids (Brosius(1988) Biotechnology 10:205-225; Wang et al (1999) J. of Fujian Agricultural University 28:43-46; Fu et. al. (1988) Chem. Abstracts 34:415-418). The expression of the dominant negative MMR gene will be turned on to select for hypermutable microbes with new output traits. Next, the expression of the dominant negative dominant negative MMR allele is rapidly turned off to reconstitute a genetically stable strain that produces a new output trait of commercial interest. The resulting microbe is now useful as a stable strain that can be applied to various commercial applications, depending upon the selection process placed upon it.

In cases where genetically deficient mismatch repair bacteria (strains such as but not limited to: M1 (mutS) and in EC2416 (mutS delta umuDC), and mutL or mutY strains) are used to derive new output traits, transgenic constructs will be used that express wild-type mismatch repair genes sufficient to complement the genetic defect and therefore restore mismatch repair activity of the host after trait selection (Grzesiuk et al. (1988) Mutagenesis 13:127-132; Bridges et al. (1997) EMBO J. 16:3349-3356; LeClerc (1996) Science 15:1208-1211; Jaworski, A. et al. (1995) Proc. Natl. Acad. Sci USA 92:11019-11023). The resulting microbe is genetically stable and can be applied to various commercial practices.

The use of over expressing foreign mismatch repair genes from human and yeast such as human PMS1 (SEQ ID NO:7), human PMS2 (SEQ ID NO:5), mouse PMS2 (SEQ ID NO:3), human MSH2 (SEQ ID NO:9), human MLH1 (SEQ ID NO:11), yeast MLH1 (SEQ ID NO:1), human MLH3 (SEQ ID NO:28), as well as the other homologs identified in other species for these encoded polypeptides etc.have been previously demonstrated to produce a dominant negative mutator phenotype in bacterial hosts (Brosh and Matson (1995) J. Bacteriol. 177:5612-5621; Studamire et al. (1998) Mol. Cell. Biol. 18:7590-7601; Alani et al. (1997) Mol. Cell. Biol.17:2436-2447). In addition, the use of bacterial strains expressing prokaryotic dominant negative MMR genes as well as hosts that have genomic defects in endogenous MMR proteins have also been previously shown to result in a dominant negative mutator phenotype (Strand et al. (1993) Nature 365:274-276; Nicolaides et al. (1998) Mol. Cell. Biol. 18:1635-1641). However, the findings disclosed here teach the use of MMR genes, including the human PMSR2 and PMSR3 gene (Nicolaides et al. (1995) Genomics 30:195-206); the related PMS134 truncated MMR gene (Nicolaides et al. (1998) Mol. Cell. Biol. 18:1635-1641); the plant mismatch repair genes (derived from Arabidopsis thaliana), ATPMS2 (SEQ ID NO:30), At PMS1 (SEQ ID NO:32), and MutS homolog (SEQ ID NO:34) and those genes that are homologous to the 134 N-terminal amino acids of the PMS2 gene which include the MutL family of MMR proteins and including the PMSR and PMS2L homologs described by Hori et al. (PMS2L8 (SEQ ID NO:36) and PMS2L9 (SEQ ID NO:38)) and Nicolaides (Nicolaides et al. (1995) Genomics 30:195-206) to create hypermutable microbes. The corresponding polypeptide sequences for the above-referenced nucleic acid sequences are as follows: yeast MLH1 (SEQ ID NO:2); mouse PMS2 (SEQ ID NO:4); human PMS2 (SEQ ID NO:6); human PMS1 (SEQ ID NO:8); human MSH2 (SEQ ID NO:10); human MLH1 (SEQ ID NO:12); PMS2-134 (SEQ ID NO:14); human MSH6 (SEQ ID NO: 16); human PMSR2 (SEQ ID NO: 18); human PMSR3 (SEQ ID NO:20); human PMSL9 (SEQ ID NO:22); human MLH3 (SEQ ID NO:29); ATPMS2 (SEQ ID NO:3 1); ATPMS1 (SEQ ID NO:33); At MutS homolog (SEQ ID NO:35); PMS2L8 (SEQ ID NO:37); and PMS2L9 (SEQ ID NO:39).

In addition, the invention provides the use of DNA mutagens in combination with MMR defective microbial hosts to enhance the hypermutable production of genetic alterations. This has not been demonstrated in the art previously as a means to accentuate MMR activity for generation of microorganisms with clinically relevant output traits such as antibiotic resistance.

In some embodiments of the invention, the bacteria cells are rendered hypermutable by introducing a chemical inhibitor of mismatch repair into the growth medium. Chemical inhibitors of mismatch repair that may be used to generate hypermutable bacterial cells include anthracene-derived compounds comprising the formula:

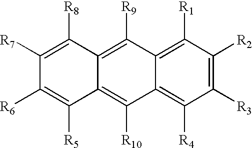

In certain preferred embodiments of the invention, the anthracene has the formula:

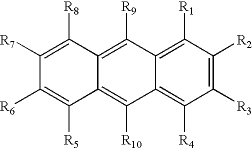

wherein R₁-R₁₀ are independently hydrogen, hydroxyl, amino, alkyl, substituted alkyl, alkenyl, substituted alkenyl, alkynyl, substituted alkynyl, O-alkyl, S-alkyl, N-alkyl, O-alkenyl, S-alkenyl, N-alkenyl,O-alkynyl, S-alkynyl, N-alkynyl, aryl, substituted aryl, aryloxy, substituted aryloxy, heteroaryl, substituted heteroaryl, aralkyloxy, arylalkyl, alkylaryl, alkylaryloxy, arylsulfonyl, alkylsulfonyl, alkoxycarbonyl, aryloxycarbonyl, guanidino, carboxy, an alcohol, an amino acid, sulfonate, alkyl sulfonate, CN, NO₂, an aldehyde group, an ester, an ether, a crown ether, a ketone, an organosulfur compound, an organometallic group, a carboxylic acid, an organosilicon or a carbohydrate that optionally contains one or more alkylated hydroxyl groups;

wherein said heteroalkyl, heteroaryl, and substituted heteroaryl contain at least one heteroatom that is oxygen, sulfur, a metal atom, phosphorus, silicon or nitrogen; and

wherein said substituents of said substituted alkyl, substituted alkenyl, substituted alkynyl, substituted aryl, and substituted heteroaryl are halogen, CN, NO₂, lower alkyl, aryl, heteroaryl, aralkyl, aralkyloxy, guanidino, alkoxycarbonyl, alkoxy, hydroxy, carboxy and amino;

and wherein said amino groups optionally substituted with an acyl group, or 1 to 3 aryl or lower alkyl groups;

or wherein any two of R₁-R₁₀ can together form a polyether;

or wherein any two of R₁-R₁₀ can, together with the intervening carbon atoms of the anthracene core, form a crown ether.

As used herein, “alkyl” refers to a hydrocarbon containing from 1 to about 20 carbon atoms. Alkyl groups may straight, branched, cyclic, or combinations thereof. Alkyl groups thus include, by way of illustration only, methyl, ethyl, propyl, isopropyl, butyl, isobutyl, cyclopentyl, cyclopentylmethyl, cyclohexyl, cyclohexylmethyl, and the like. Also included within the definition of “alkyl” are fused and/or polycyclic aliphatic cyclic ring systems such as, for example, adamantane. As used herein the term “alkenyl” denotes an alkyl group having at least one carbon-carbon double bond. As used herein the term “alkynyl” denotes an alkyl group having at least one carbon-carbon triple bond. In certain preferred embodiments of the invention, the anthracene has the formula:

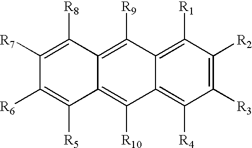

wherein R₁-R₁₀ are independently hydrogen, hydroxyl, amino, alkyl, substituted alkyl, alkenyl, substituted alkenyl, alkynyl, substituted alkynyl, O-alkyl, S-alkyl, N-alkyl, O-alkenyl, S-alkenyl, N-alkenyl,O-alkynyl, S-alkynyl, N-alkynyl, aryl, substituted aryl, aryloxy, substituted aryloxy, heteroaryl, substituted heteroaryl, aralkyloxy, arylalkyl, alkylaryl, alkylaryloxy, arylsulfonyl, alkylsulfonyl, alkoxycarbonyl, aryloxycarbonyl, guanidino, carboxy, an alcohol, an amino acid, sulfonate, alkyl sulfonate, CN, NO₂, an aldehyde group, an ester, an ether, a crown ether, a ketone, an organosulfur compound, an organometallic group, a carboxylic acid, an organosilicon or a carbohydrate that optionally contains one or more alkylated hydroxyl groups;

wherein said heteroalkyl, heteroaryl, and substituted heteroaryl contain at least one heteroatom that is oxygen, sulfur, a metal atom, phosphorus, silicon or nitrogen; and

wherein said substituents of said substituted alkyl, substituted alkenyl, substituted alkynyl, substituted aryl, and substituted heteroaryl are halogen, CN, NO₂, lower alkyl, aryl, heteroaryl, aralkyl, aralkyloxy, guanidino, alkoxycarbonyl, alkoxy, hydroxy, carboxy and amino;

and wherein said amino groups optionally substituted with an acyl group, or 1 to 3 aryl or lower alkyl groups;

or wherein any two of R₁-R₁₀ can together form a polyether;

or wherein any two of R₁-R₁₀ can, together with the intervening carbon atoms of the anthracene core, form a crown ether.

As used herein, “alkyl” refers to a hydrocarbon containing from 1 to about 20 carbon atoms. Alkyl groups may straight, branched, cyclic, or combinations thereof. Alkyl groups thus include, by way of illustration only, methyl, ethyl, propyl, isopropyl, butyl, isobutyl, cyclopentyl, cyclopentylmethyl, cyclohexyl, cyclohexylmethyl, and the like. Also included within the definition of “alkyl” are fused and/or polycyclic aliphatic cyclic ring systems such as, for example, adamantane. As used herein the term “alkenyl” denotes an alkyl group having at least one carbon-carbon double bond. As used herein the term “alkynyl” denotes an alkyl group having at least one carbon-carbon triple bond.

In some embodiments, the anthracene has the formula:

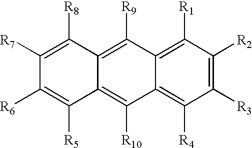

wherein R₁-R₁₀ are independently hydrogen, hydroxyl, amino, alkyl, substituted alkyl, alkenyl, substituted alkenyl, alkynyl, substituted alkynyl, O-alkyl, S-alkyl, N-alkyl, O-alkenyl, S-alkenyl, N-alkenyl, O-alkynyl, S-alkynyl, N-alkynyl, aryl, substituted aryl, aryloxy, substituted aryloxy, heteroaryl, substituted heteroaryl, aralkyloxy, arylalkyl, alkylaryl, alkylaryloxy, arylsulfonyl, alkylsulfonyl, alkoxycarbonyl, aryloxycarbonyl, guanidino, carboxy, an alcohol, an amino acid, sulfonate, alkyl sulfonate, CN, NO₂, an aldehyde group, an ester, an ether, a crown ether, a ketone, an organosulfur compound, an organometallic group, a carboxylic acid, an organosilicon or a carbohydrate that optionally contains one or more alkylated hydroxyl groups;

wherein said heteroalkyl, heteroaryl, and substituted heteroaryl contain at least one heteroatom that is oxygen, sulfur, a metal atom, phosphorus, silicon or nitrogen; and

wherein said substituents of said substituted alkyl, substituted alkenyl, substituted alkynyl,

substituted aryl, and substituted heteroaryl are halogen, CN, NO₂, lower alkyl, aryl, heteroaryl, aralkyl, aralkyloxy, guanidino, alkoxycarbonyl, alkoxy, hydroxy, carboxy and amino;

and wherein said amino groups optionally substituted with an acyl group, or 1 to 3 aryl or lower alkyl groups.

Examples of such anthracenes include, but are not limited to 1,2-dimethylanthracene, 9,10-dimethyl anthracene, 7,8-dimethylanthracene, 9,10-diphenylanthracene, 9,10-dihydroxymethylanthracene, 9-hydroxymethyl-10-methylanthracene, dimethylanthracene-1,2-diol, 9-hydroxymethyl-10-methylanthracene-1,2-diol, 9-hydroxymethyl-10-methylanthracene-3,4-diol, and 9,10-di-m-tolyanthracene.

As used herein the term “anthracene” refers to the compound anthracene. However, when referred to in the general sense, such as “anthracenes,” “an anthracene” or “the anthracene,” such terms denote any compound that contains the fused triphenyl core structure of anthracene, i.e.,

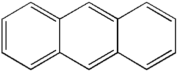

regardless of extent of substitution.

In some embodiments, the alkyl, alkenyl, alkynyl, aryl, aryloxy, and heteroaryl substituent groups described above may bear one or more further substituent groups; that is, they may be “substituted”. In some preferred embodiments these substituent groups can include halogens (for example fluorine, chlorine, bromine and iodine), CN, NO₂, lower alkyl groups, aryl groups, heteroaryl groups, aralkyl groups, aralkyloxy groups, guanidino, alkoxycarbonyl, alkoxy, hydroxy, carboxy and amino groups. In addition, the alkyl and aryl portions of aralkyloxy, arylalkyl, arylsulfonyl, alkylsulfonyl, alkoxycarbonyl, and aryloxycarbonyl groups also can bear such substituent groups. Thus, by way of example only, substituted alkyl groups include, for example, alkyl groups fluoro-, chloro-, bromo- and iodoalkyl groups, aminoalkyl groups, and hydroxyalkyl groups, such as hydroxymethyl, hydroxyethyl, hydroxypropyl, hydroxybutyl, and the like. In some preferred embodiments such hydroxyalkyl groups contain from 1 to about 20 carbons.

As used herein the term “aryl” means a group having 5 to about 20 carbon atoms and which contains at least one aromatic ring, such as phenyl, biphenyl and naphthyl. Preferred aryl groups include unsubstituted or substituted phenyl and naphthyl groups. The term “aryloxy” denotes an aryl group that is bound through an oxygen atom, for example a phenoxy group.

In general, the prefix “hetero” denotes the presence of at least one hetero (i.e., non-carbon) atom, which is in some preferred embodiments independently one to three O, N, S, P, Si or metal atoms. Thus, the term “heteroaryl” denotes an aryl group in which one or more ring carbon atom is replaced by such a heteroatom. Preferred heteroaryl groups include pyridyl, pyrimidyl, pyrrolyl, furyl, thienyl, and imidazolyl groups.

The term “aralkyl” (or “arylalkyl”) is intended to denote a group having from 6 to 15 carbons, consisting of an alkyl group that bears an aryl group. Examples of aralkyl groups include benzyl, phenethyl, benzhydryl and naphthylmethyl groups.

The term “alkylaryl” (or “alkaryl”) is intended to denote a group having from 6 to 15 carbons, consisting of an aryl group that bears an alkyl group. Examples of aralkyl groups include methylphenyl, ethylphenyl and methylnaphthyl groups.

The term “arylsulfonyl” denotes an aryl group attached through a sulfonyl group, for example phenylsulfonyl. The term “alkylsulfonyl” denotes an alkyl group attached through a sulfonyl group, for example methylsulfonyl.

The term “alkoxycarbonyl” denotes a group of formula —C(═O)—O—R where R is alkyl, alkenyl, or aLkynyl, where the alkyl, alkenyl, or alkynyl portions thereof can be optionally substituted as described herein.

The term “aryloxycarbonyl” denotes a group of formula —C(═O)—O—R where R is aryl, where the aryl portion thereof can be optionally substituted as described herein.

The terms “arylalkyloxy” or “aralkyloxy” are equivalent, and denote a group of formula —O—R′—R″, where R′ is R is alkyl, alkenyl, or alkynyl which can be optionally substituted as described herein, and wherein R″ denotes a aryl or substituted aryl group.

The terms “alkylaryloxy” or “alkaryloxy” are equivalent, and denote a group of formula —O—R′—R″, where R′ is an aryl or substituted aryl group, and R″ is alkyl, alkenyl, or alkynyl which can be optionally substituted as described herein.

As used herein, the term “aldehyde group” denotes a group that bears a moiety of formula —C(═O)—H. The term “ketone” denotes a moiety containing a group of formula —R—C(═O)—R═, where R and R= are independently alkyl, alkenyl, alkynyl, aryl, heteroaryl, aralkyl, or alkaryl, each of which may be substituted as described herein.

As used herein, the term “ester” denotes a moiety having a group of formula —R—C(═O)—O—R= or —R—O—C(═O)—R=where R and R= are independently alkyl, alkenyl, alkynyl, aryl, heteroaryl, aralkyl, or alkaryl, each of which may be substituted as described herein.

The term “ether” denotes a moiety having a group of formula —R—O—R= or where R and R= are independently alkyl, alkenyl, alkynyl, aryl, heteroaryl, aralkyl, or alkaryl, each of which may be substituted as described herein.

The term “crown ether” has its usual meaning of a cyclic ether containing several oxygen atoms. As used herein the term “organosulfur compound” denotes aliphatic or aromatic sulfur containing compounds, for example thiols and disulfides. The term “organometallic group” denotes an organic molecule containing at least one metal atom.

The term “organosilicon compound” denotes aliphatic or aromatic silicon containing compounds, for example alkyl and aryl silanes.

The term “carboxylic acid” denotes a moiety having a carboxyl group, other than an amino acid.

As used herein, the term “amino acid” denotes a molecule containing both an amino group and a carboxyl group. In some preferred embodiments, the amino acids are α-, β-, γ- or δ-amino acids, including their stereoisomers and racemates. As used herein the term “L-amino acid” denotes an a-amino acid having the L configuration around the α-carbon, that is, a carboxylic acid of general formula CH(COOH)(NH₂)-(side chain), having the L-configuration. The term “D-amino acid” similarly denotes a carboxylic acid of general formula CH(COOH)(NH₂)-(side chain), having the D-configuration around the α-carbon. Side chains of L-amino acids include naturally occurring and non-naturally occurring moieties. Non-naturally occurring (i.e., unnatural) amino acid side chains are moieties that are used in place of naturally occurring amino acid side chains in, for example, amino acid analogs. See, for example, Lehninger, Biochemistry, Second Edition, Worth Publishers, Inc, 1975, pages 72-77, incorporated herein by reference. Amino acid substituents may be attached through their carbonyl groups through the oxygen or carbonyl carbon thereof, or through their amino groups, or through functionalities residing on their side chain portions.

In some embodiments of the methods of the invention, the cells are made hypermutable using ATP analogs capable of blocking ATPase activity required for MMR. MMR reporter cells are screened with ATP compound libraries to identify those compounds capable of blocking MMR in vivo. Examples of ATP analogs that are useful in blocking MMR activity include, but are not limited to, nonhydrolyzable forms of ATP such as AMP-PNP and ATP[gamma]S block the MMR activity (Galio, L. et al. (1999) Nucl. Acids Res. 27:2325-2331; Allen, D. J. et al. (1997) EMBO J. 16:4467-4476; Bjornson K. P. et al. (2000) Biochem. 39:3176-3183). The ATPase inhibitors inhibit MMR and the cells become hypermutable as a result.

In other embodiments of the methods of the invention, the cells are made hypermutable using nuclease inhibitors that are able to block the exonuclease activity of the MMR biochemical pathway. MMR reporter cells are screened with nuclease inhibitor compound libraries to identify compounds capable of blocking MMR in vivo. Examples of nuclease inhibitors that are useful in blocking MMR activity include, but are not limited to analogs of N-Ethylmaleimide, an endonuclease inhibitor (Huang, Y. C., et.al. (1995) Arch. Biochem. Biophys. 316:485), heterodimeric adenine-chain-acridine compounds, exonulcease III inhibitors (Belmont P, et.al., Bioorg Med Chem Lett (2000) 10:293-295), as well as antibiotic compounds such as Heliquinomycin, which have helicase inhibitory activity (Chino, M, et.al. J. Antibiot. (Tokyo) (1998) 51:480-486). The nuclease inhibitors inhibit MMR and the cells become hypermutable as a result.

In other embodiments of the methods of the invention, the cells are made hypermutable using DNA polymerase inhibitors that are able to block the polymerization required for mismatch-mediated repair. MMR reporter cells are screened with DNA polymerase inhibitor compound libraries to identify those compounds capable of blocking MMR in vivo. Examples of DNA polymerase inhibitors that are useful in blocking MMR activity include, but are not limited to, analogs of actinomycin D (Martin, S. J., et.al. (1990) J. Immunol. 145:1859), Aphidicolin (Kuwakado, K. et.al. (1993) Biochem. Pharmacol. 46:1909) 1-(2′-Deoxy-2′-fluoro-beta-L-arabinofuranosyl)-5-methyluracil (L-FMAU) (Kukhanova M, et.al., Biochem Pharmacol (1998) 55:1181-1187), and 2′,3′-dideoxyribonucleoside 5′-triphosphates (ddNTPs) (Ono, K., et.al., Biomed Pharmacother (1984) 38:382-389). The polymerase inhibitors inhibit MMR and the cells become hypermutable as a result.

Bacterial cells rendered hypermutable using chemical inhibitors of MMR may be made genetically stable when the desired phenotype is obtained by removing the MMR inhibitory molecule.

In certain embodiments, the bacterial cells are made hypermutable by introducing plamids that generate antisense messages wherein the antisense RNA specifically bind to MMR genes and prevent efficient expression of MMR proteins. Preferably, the antisense transcripts are at least 12 nucleotides in length and, more preferably are at least 20, 30, 40, 50 nucleotides or more in length. The antisense transcripts specifically bind to regions of the MMR gene to inhibit expression. Preferably, the antisense transcripts specifically bind to regulatory regions of the MMR gene such as to the MMR promoter region, Kozak consensus sequences, and the like. As used herein, “specifically bind” refers to association of nucleic acid strands forming complementary base pairing in Watson-Crick arrangement, allowing for mismatches such that 100% complementarity is not required. In general, the complementarity will be about 85%, 90%, 95% or more. Plasmids that may be used to express an antisense MMR transcript include any vector generally known in the art to express antisense transcripts, such as for example, those found in Qian Y. et al. (1998) Mutat. Res. 418(2-3):61-71. The above disclosure generally describes the present invention. A more complete understanding can be obtained by reference to the following specific examples that will be provided herein for purposes of illustration only, and are not intended to limit the scope of the invention.

### EXAMPLE1

### Generation of MMR Defective Bacteria

Bacterial expression constructs were prepared using the human PMS2 related gene (hPMSR3) (Nicolaides et al. (1995) Genomics 30:195-206) and the human PMS134 cDNA (Nicolaides et al. (1998) Mol. Cell. Biol. 18:1635-1641), both of which are capable of inactivating MMR activity and thereby increase the overall frequency of genomic hypermutation. Moreover, the use of regulatable expression vectors will allow for suppression of dominant negative MMR alleles and restoration of the MMR pathway and genetic stability in hosts cells (Brosius, J. (1988) Biotechnology 10:205-225). For these studies, a plasmid encoding the hPMS134 cDNA was altered by polymerase chain reaction (PCR). The 5′ oligonucleotide has the following sequence: 5′-acg cat atg gag cga gct gag agc tcg agt-3′(SEQ ID NO:23) that includes the NdeI restriction site (cat atg). The 3′-oligonucleotide has the following sequence: 5′-gaa ttc tta tca cgt aga atc gag acc gag gag agg gtt agg gat agg ctt acc agt tcc aac ctt cgc cga tgc-3′ (SEQ ID NO:24) that includes an EcoRi site (gaattc) and the 14 amino acid epitope for the V5 antibody. The oligonucleotides were used for PCR under standard conditions that included 25 cycles of PCR (95° C. for 1 minute, 55° C. for 1 minute, 72° C. for 1.5 minutes for 25 cycles followed by 3 minutes at 72° C. PCR fragment was purified by gel electrophoresis and cloned into pTA2.1 (InVitrogen) by standard cloning methods (Sambrook et al., MOLECULAR CLONING: A LABORATORY MANUAL, THIRD EDITION, 2001), creating the plasmid pTA2.1-hPMS134. The pTA2.1-hPMS134 plasmid was digested with the restriction enzyme EcoRi to release the insert (there are two EcoRi restriction sites in the multiple cloning site of pTA2.1 that flank the insert) and the fragment was end-filled using Klenow fragment and dNTPs. Next, the fragment was gel purified, digested with NdeI, and inserted in pT7-Ea (which had been digested with NdeI and BamHI, end-filledusing Klenow, and phosphatase treated). The new plasmid was designated pT7-Ea-hPMS134.

The following strategy, similar to that described above to clone human PMS134, was used to construct an expression vector for the human related gene PMSR3. First, the hPMSR3 fragment was amplified by PCR to introduce two restriction sites: an NdeI restriction site at the 5′- end, and an Eco RI site at the 3′-end of the fragment. The 5′-oligonucleotide that was used for PCR has the following sequence: 5′-acg cat atg tgt cct tgg cgg cct aga-3′ (SEQ ID NO:25) that includes the NdeI restriction site (CAT ATG). The 3′-oligonucleotide used for PCR has the following sequence: 5′-gaa ttc tta tta cgt aga atc gag acc gag gag agg gtt agg gat agg ctt acc cat gtg tga tgt ttc aga gct-3′ (SEQ ID NO:26) that includes an EcoRi site (gaattc) and the V5 epitope to allow for antibody detection. The plasmid that contained human PMSR3 in pBluescript SK (Nicolaides et al. (1995) Genomics 30:195-206) was used as the PCR target with the hPMS2-specific oligonucleotides above. Following 25 cycles of PCR (95° C. for 1 minute, 55° C. for 1 minute, 72° C for 1.5 minutes for 25 cycles followed by 3 minutes at 72° C.). The PCR fragment was purified by gel electrophoresis and cloned into pTA2.1 (InVitrogen) by standard cloning methods (Sambrook et al., MOLECULAR CLONING: A LABORATORY MANUAL, THIRD EDITION, 2001), creating the plasmid pTA2.1-hR3. The pTA2.1-hR3 plasmid was next digested with the restriction enzyme EcoRI to release the insert (there are two EcoRI restriction sites in the multiple cloning site of pTA2.1 that flank the insert) and the fragment was end-filled using Klenow fragment and dNTPs. Then, the fragment was gel purified, digested with NdeI, and inserted in pT7-Ea (which had been digested with NdeI and BamHI, end-filled using Klenow, and phosphatase treated). The new plasmid was designated pT7-Ea-hR3.

BL21 cells harbor an additional expression vector for the lysozyme protein, which has been demonstrated to bind to the T7 polymerase in situ; this results in a bacterial strain that has very low levels of T7 polymerase expression. However, upon addition of the inducer isopropyl-beta-D-thiogalactopyranoside (IPTG), the cells express high-levels of T7 polymerase due to the IPTG-inducible element that drives expression of the polymerase that is resident within the genome of the BL21 cells (Studier et al. (1991) J. Mol. Biol. 219(1):37-44). The BL21 cells are chloramphenicol resistant due to the plasmid that expresses lysozyme within the cell. To introduce the pT7-hPMS134 or the pT7-hPMSR3 genes into BL21 cells, the cells were made competent by incubating the cells in ice cold 50 mM CaCl₂ for 20 minutes, followed by concentrating the cells and adding super-coiled plasmid DNA as describe ( Sambrook et al., MOLECULAR CLONING: A LABORATORY MANUAL, THIRD EDITION, Cold Spring Harbor Laboratory Press, 2001). Ampicillin resistant BL21 were selected on LB-agar plates [5% yeast extract, 10% bactotryptone, 5% NaCl, 1.5% bactoagar, pH 7.0 (Difco)] plates containing 25 μg/ml chloramphenicol and 100 μg/ml ampicillin. The next day, bacterial colonies were selected and analyzed by restriction endonuclease digestion and sequence analysis for plasmids containing an intact pTACPMS134 or pTAC empty plasmid.

In addition to constructing a V5-epitope tagged PMS134 construct, we also constructed and tested a non-epitope tagged version. This was prepared to demonstrate that the epitope tag did not cause the alteration of the dominant-negative phenotype that PMS134 has on mismatch repair activity. For these studies, a BamHI restriction fragment containing the hPMS134 cDNA was filled-in using Klenow fragment and then sub-cloned into a Klenow-filled, blunt-ended NdeI-XhoI site of the pTACLAC expression vector (which contains the IPTG-inducible bacterial TAC promoter and ampicillin resistance gene as selectable marker). The NdeI-XhoI cloning site is flanked by the TACLAC promoter that contains the LacI repressor site followed by a Shine-Dalgarno ribosome-binding site at the 5′ flanking region and the T1T2 ribosomal RNA terminator in the 3′ flanking region. The TACLAC vector also contains the LacI gene, which is constitutively expressed by the TAC promoter.

DH10OB bacterial cells containing the pBCSK vector (Stratagene), which constitutively expresses the, β-galactosidase gene and contains the chloramphenicol resistance marker for selection, were made competent via the CaCl₂ method (Sambrook, et al. MOLECULAR CLONING: A LABORATORY MANUAL, Cold Spring Harbor Laboratory Press, 1982). This vector turns bacterial cells blue when grown in the presence of IPTG and X-gal that aids in the detection of bacterial colonies. Competent cells were transfected with the pTAC empty vector or the pTACPMS134 vector following the heat-shock protocol. Transfected cultures were plated onto LB-agar [5% yeast extract, 10% bactotryptone, 5% NaCl, 1.5% bactoagar, pH 7.0 (Difco)] plates containing 25 μg/ml chloramphenicol and 100 μg/ml ampicillin. The next day, bacterial colonies were selected and analyzed by restriction endonuclease digestion and sequence analysis for plasmids containing an intact pTACPMS134 or pTAC empty plasmid. Ten clones of each bacteria containing correct empty or PMS134 inserts were then grown to confluence overnight in LB media (5% yeast extract, 10% bactotryptone, 5% NaCl, pH 7.0) containing 10 μg/ml chloramphenicol and 50 μg/ml ampicillin. The next day TAC empty or pTACPMS134 cultures were diluted 1:4 in LB medium plus 50 μM IPTG (Gold Biotechnology) and cultures were grown for 12 and 24 hours at 37° C. After incubation, 50 μl aliquots were taken from each culture and added to 150 μls of 2× SDS buffer and cultures were analyzed for PMS134 protein expression by western blot.

Western blots were carried out as follows: 50 μls of each PMS134 or empty plasmid culture was directly lysed in 2× lysis buffer (60 mM Tris, pH 6.8, 2% SDS, 10% glycerol, 0.1 M 2-mercaptoethanol, 0.001% bromophenol blue) and samples were boiled for 5 minutes. Lysate proteins were separated by electrophoresis on 4-20% Tris glycine gels (Novex). Gels were electroblotted onto Immobilon-P (Millipore) in 48 mM Tris base, 40 mM glycine, 0.0375% SDS, 20% methanol and blocked overnight at 4° C. in Tris-buffered saline plus 0.05% Tween-20 and 5% condensed milk. Filters were probed with a rabbit polyclonal antibody generated against the N-terminus of the human PMS2 polypeptide (Santa Cruz), which is able to recognize the PMS134 polypeptide (Su et al. (1988) J. Biol. Chem. 263:6829-6835), followed by a secondary goat anti-rabbit horseradish peroxidase-conjugated antibody. Alternatively, blots were probed with an anti-V5 monoclonal antibody followed by a secondary goat anti-mouse horseradish peroxidase-conjugated antibody. After incubation with the secondary antibody, blots are developed using chemiluminescence (Pierce) and exposed to film to measure PMS134 expression.

For induction of PMS gene product, 5 ml cultures of Luria Broth (LB) plus 50 μg/ml ampicillin were inoculated from glycerol stocks of the transformants pT7Ea (BL21), pT7PMS134/V5 (BL21), or pT7PMSR3 (BL21) and grown overnight at 37° C. with shaking. 200 μl of each overnight culture was inoculated in 20 ml (1:100) fresh LB broth plus ampicillin and grown to an OD₆₀₀ of 0.6. 20 μl of 100 mM IPTG (final concentration 0.1 mM) was added and cultures were grown overnight. Western analysis confirmed the presence of inducible PMS expression in the presence of inducer molecule (not shown).

### EXAMPLE 2

### Generation of Antibiotic Resistant Bacteria

To demonstrate the ability to produce antibiotic resistant bacterial strains by inhibiting MMR, 10⁷ bacterial cells expressing either the vector (pT7Ea) or pT7PMS134/V5 were inoculated into 5 ml LB broth plus the appropriate antibiotic concentrations as shown below (Table 1) and grown overnight at 37° C. with shaking. Antibiotic concentrations were based on 0.5× the minimum inhibitory concentrations (MIC) observed to inhibit the growth of bacteria constitutively expressing the mar operon (Goldman et al. (1996) Antimicrobial Agents Chemother. 40: 1266-1269). Titration analysis found the following amounts to be effective in inhibiting bacterial growth in the presence of various compounds.

TABLE 1
Half minimum inhibitory concentrations (MIC) on BL21 cells.

| DRUG | 0.5X MIC (μg/ml) |
|---|---|
| Tetracycline | 4.70 |
| Nalidixic Acid | 7.10 |
| Ofloxacin | 0.13 |
| Norfloxacin | 0.13 |
| Vancomycin | 250.0 |

The next day, cultures were analyzed for cell growth in the presence or absence of antibiotics. Table 2 summarizes typical data from these studies. No growth was observed in bacterial control cells (pT7Ea), which had OD levels similar to blank culture. In contrast, significant culture growth was observed in pT7PMS134V5 and pT7PMSR3 (not shown) cultures grown in all antibiotics tested (Table 2)

TABLE 2
Overnight Growth of Drug Resistant Mutants
Expressing the PMS2-134.

| | pT7Ea | | pT7PMS134V5 | |
|---|---|---|---|---|
| Drug | growth | Cell # | growth | Cell # (X10⁹) |
| Tetracycline | − | 0 | + | 1.10 |
| Nalidixic Acid | − | 0 | + | 0.97 |
| Ofloxacin | − | 0 | + | 1.20 |
| Norfloxacin | − | 0 | + | 1.40 |
| Vancomycin | − | 0 | + | ND |

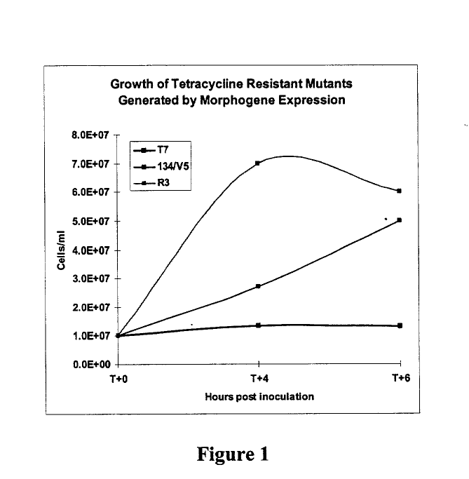

To test the stability of antibiotic resistance, cells were replated and followed for growth in the presence of 1× MIC concentration of antibiotic. Table 3 shows an example in which bacterial cells were inoculated at 1×10⁷ cells/ml and grown for 6 hours in the presence of tetracycline (Tet). As shown in FIG. 1, pT7Ea control culture did not grow in the presence of Tet while pT7PMS134 and pT7PMSR3 cultures resistant to Tet grew to confluence at time 4 hours after inoculation. These data demonstrate the ability to generate antibiotic resistant cultures by blocking MMR and reestablishing genetically stable cultures that can be used for gene discovery.

### EXAMPLE 3

### Genomic Analysis of Antibiotic Resistant Bacteria and Target Discovery.

The ability to generate a wide degree of genomic mutation in MMR defective bacteria allow for the rapid analysis of the AR host's genome in comparison to the wild-type strain. While many methods for mutation analysis exist that are know by those skilled in the art, several approaches exist that allow for the screening of unknown genes as well as those that exist which are capable of screening for mutants within “candidate” genes that are capable of conferring an antiobiotic resistant phenotype. One such method includes the use of in vitro-coupled-translation strategies, which is a rapid method that is used to screen for mutations that result in truncated polypeptides (Liu et al. (1996) Nat. Med. 2:169-174; Nicolaides et al. (1994) Nature 371: 75-80; Papadopoulos et al. (1994) Science 263:1625-1629; Nicolaides et al. (1998) Mol. Cell. Biol. 18:1635-1641; Alekshun, M. N. and S. B. Levy (1999) J. Bacteriol. 181:3303-3306).

In vitro Transcription-coupled-translation

Linear DNA fragments containing candidate gene sequences were prepared by PCR, incorporating sequences for in vitro transcription and translation in the sense primer. The sense primer contains the leader sequence 5′-tttaatacgactcactatagggagaccaccatggnnn nnn nnn nnn nnn-3′ (SEQ ID NO:27) where the series of “n” nuclsotides indicates sequence corresponding to the first 5 codons. The antisense primer consists of nucleotide sequences surrounding and including the natural stop codon of the gene. DNA fragments are PCR amplified using buffers and condions as described (Nicolaides et al. (1995) Genomics 30:195-206). Two to five microliters of whole bacteria are added to the PCR reaction mix and reactions are carried out at 95° C. for 1 minute for one cycle followed by thirty cycles at 95° C. for 30 sec, 52° C. for 1 minute and 72° C. for 2 minutes. PCR products are then directly added to a rabbiti reticulolysate mixture to carry out transcription-coupled-translation (Promega). The reaction mixtures were supplemented with [³⁵S]-methionine for detection purposes. Translation reactions are incubated for 2 hours. After the reaction is complete, an equal volume of 2× SDS lysis buffer is added to the samples, and the samples are boiled and then loaded onto 12% NuPAGE gels (Novex). Gels are run at 150V, dried and exposed to autoradiography. Products that are smaller than the expected molecular weight of the wild-type protein (as compared to the control samples) are then determined to be mutant and DNA fragments are sequenced to confirm the presence of a frame-shift/nonsense mutation. This approach has been used to identify mutations in bacterial genes that have been previously been reported to produce antibiotic resistance in bacteria.

Discussion

The results described above lead to several conclusions. The inhibition of MMR results in an increase in hypermutability in bacteria. This activity is due to the inhibition of MMR biochemical activity in these hosts. This invention provides a novel method of producing antibiotic resistant strains for target discovery and the rational design of novel anti-microbial agents to each target identified by generating AR bacteria through the inhibition of mismatch repair.

The disclosures of the following references, as well as the references cited herein, are hereby incorporated by reference in their entirety.

References

1. Attfield, P. V. and Pinney, R. J. 1985. Elimination of multicopy plasmid R6K by bleomycin. Antimicrob. Agents Chemother. 27(6):985-988.

2. Baker S. M., Bronner, C. E., Zhang, L., Plug, A. W., Robatez, M., Warren, G., Elliott, E. A., Yu, J., Ashley, T., Arnheim, N., Bradley, N., Flavell, R. A., and Liskay, R. M. 1995. Male defective in the DNA mismatch repair gene PMS2 exhibit abnormal chromosome synapsis in meiosis. Cell 82:309-319.

3. Berry, A. 1996. Improving production of aromatic compounds in Escherichia coli by metabolic engineering. Trends Biotechnol. 14(7):250-256.

4. Bronner, C. E., Baker, S. M., Morrison, P. T., Warren, G., Smith, L. G., Lescoe, M. K., Kane, M., Earabino, C., Lipford, J., Lindblom, A., Tannergard, P., Bollag, R. J., Godwin, A., R., Ward, D. C., Nordenskjold, M., Fishel, R., Kolodner, R., and Liskay, R. M. 1994. Mutation in the DNA mismatch repair gene homologue hMLH1 is associated with hereditary non-polyposis colon cancer. Nature 368:258-261.

5. de Wind N., Dekker, M., Berns, A., Radman, M., and Riele, H. T. 1995. Inactivation of the mouse Msh2 gene results in mismatch repair deficiency, methylation tolerance, hyperrecombination, and predisposition to cancer. Cell 82:321-300.

6. Drummond, J. T., Li, G. M., Longley, M. J., and Modrich, P. 1995. Isolation of an hMSH2-p160 heterodimer that restores mismatch repair to tumor cells. Science 268:1909-1912.

7. Drummond, J. T., Anthoney, A., Brown, R., and Modrich, P. 1996. Cisplatin and adriamycin resistance are associated with MutLa and mismatch repair deficiency in an ovarian tumor cell line. J.Biol.Chem. 271:9645-19648.

8. Edelmann, W., Cohen, P. E., Kane, M., Lau, K., Morrow, B., Bennett, S., Umar, A., Kunkel, T., Cattoretti, G., Chagnatti, R., Pollard, J. W., Kolodner, R. D., and Kucherlapati, R. 1996. Meiotic pachytene arrest in MLH1-deficient mice. Cell 85:1125-1134.

9. Fishel, R., Lescoe, M., Rao, M. R. S., Copeland, N. J., Jenkins, N. A., Garber, J., Kane, M., and Kolodner, R. 1993. The human mutator gene homolog MSH2 and its association with hereditary nonpolyposis colon cancer. Cell 7:1027-1038.

10. Fu, K. P., Grace, M. E., Hsiao, C. L., and Hung, P. P. 1988. Elinination of antibiotic-resistant plasmids by quinolone antibiotics. Chemotherapy 34(5):415-418.

11. Leach, F. S., Nicolaides, N. C, Papadopoulos, N., Liu, B., Jen, J., Parsons, R., Peltomaki, P., Sistonen, P., Aaltonen, L. A., Nystrom-Lahti, M., Guan, X. Y., Zhang, J., Meltzer, P. S., Yu, J. W., Kao, F. T., Chen, D. J., Cerosaletti, K. M., Fournier, R. E. K., Todd, S., Lewis, T., Leach R. J., Naylor, S. L., Weissenbach, J., Mecklin, J. P., Jarvinen, J. A., Petersen, G. M., Hamilton, S. R., Green, J., Jass, J., Watson, P., Lynch, H. T., Trent, J. M., de la Chapelle, A., Kinzler, K. W., and Vogelstein, B. 1993. Mutations of a mutS homolog in hereditary non-polyposis colorectal cancer. Cell 75:1215-1225.

12. Li, G. -M., and Modrich, P. 1995. Restoration of mismatch repair to nuclear extracts of H6 colorectal tumor cells by a heterodimer of human mutL homologs. Proc. Natl. Acad. Sci. USA 92:1950-1954.

13. Liu, B., Parsons, R., Papadopoulos, N., Nicolaides, N. C., Lynch, H. T., Watson, P., Jass, J. R., Dunlop, M., Wyllie, A., Peltomaki, P., de la Chapelle, A., Hamilton, S. R., Vogelstein, B., and Kinzler, K. W. 1996. Analysis of mismatch repair genes in hereditary non-polyposis colorectal cancer patients. Nat. Med. 2:169-174.

14. Modrich, P. 1994. Mismatch repair, genetic stability, and cancer. Science 266:1959-1960.

15. Nicolaides, N. C., Gualdi, R., Casadevall, C., Manzella, L., and Calabretta, B. 1991. Positive autoregulation of c-myb expression via MYB binding in the 5′ flanking region of the human c-myb gene. Mol. Cell. Biol. 11:6166-6176.

16. Nicolaides, N. C., Correa, I., Casadevall, C., Travali, S., Soprano, K. J., and Calabretta, B. 1992. The Jun family members, c-JUN and JUND, transactivate the human c-myb promoter via an Ap1 like element. J. Biol. Chem. 267, 19665-19672.

17. Nicolaides, N. C., Papadopoulos, N., Liu, B., Wei, Y. F., Carter, K. C., Ruben, S. M., Rosen, C. A., Haseltine, W. A., Fleischmann, R. D., Fraser, C. M., Adams, M. D., Venter, C. J., Dunlop, M. G., Hamilton, S. R., Petersen, G. M., de la Chapelle, A., Vogelstein, B., and kinzler, K. W. 1994. Mutations of two PMS homologs in hereditary nonpolyposis colon cancer. Nature 371: 75-80.

18. Nicolaides N. C., Kinzler, K. W., and Vogelstein, B. 1995. Analysis of the 5′ region of PMS2 reveals heterogenous transcripts and a novel overlapping gene. Genomics 29:329-334.

19. Nicolaides, N. C., Carter, K. C., Shell, B. K., Papadopoulos, N., Vogelstein, B., and Kinzler, K. W. 1995. Genomic organization of the human PMS2 gene family. Genomics 30:195-206.

20. Nicolaides, N. C., Palombo, F., Kinzler, K. W., Vogelstein, B., and Jiricny, J. 1996. Molecular cloning of the N-terminus of GTBP. Genomics 31:395-397.

21. Palombo, F., Hughes, M., Jiricny, J., Truong, O., Hsuan, J. 1994. Mismatch repair and cancer. Nature 36:417.

22. Palombo, F., Gallinari, P., Iaccarino, I., Lettieri, T., Hughes, M. A., Truong, O., Hsuan, J. J., and Jiricny, J. 1995. GTBP, a 160-kilodalton protein essential for mismatch-binding activity in human cells. Science 268:1912-1914.

23. Pang, Q., Prolla, T. A., and Liskay, R. M. 1997. Functional domains of the Saccharomyces cerevisiae Mlh1p and Pms1p DNA mismatch repair proteins and their relevance to human hereditary nonpolyposis colorectal cancer-associated mutations. Mol. Cell. Biol. 17:4465-4473.

24. Papadopoulos, N., Nicolaides, N. C., Wei, Y. F., Carter, K. C., Ruben, S. M., Rosen, C. A., Haseltine, W. A., Fleischmann, R. D., Fraser, C. M., Adams, M. D., Venter, C. J., Dunlop, M. G., Hamilton, S. R., Petersen, G. M., de la Chapelle, A., Vogelstein, B., and kinzler, K. W. 1994. Mutation of a mutL homolog is associated with hereditary colon cancer. Science 263:1625-1629.

25. Parsons, R., Li, G. M., Longley, M. J., Fang, W. H., Papadopolous, N., Jen, J., de la Chapelle, A., Kinzler, K. W., Vogelstein, B., and Modrich, P. 1993. Hypermutability and mismatch repair deficiency in RER⁺tumor cells. Cell 75:1227-1236.

26. Parsons, R., Li, G. M., Longley, M., Modrich, P., Liu, B., Berk, T., Hamilton, S. R., Kinzler, K. W., and Vogelstein, B. 1995. Mismatch repair deficiency in phenotypically normal human cells. Science 268:738-740.

27. Perucho, M. 1996. Cancer of the microsattelite mutator phenotype. Biol Chem. 377:675-684.

28. Prolla, T. A, Pang, Q., Alani, E., Kolodner, R. A., and Liskay, R. M. 1994. MLH1, PMS1, and MSH2 Interaction during the initiation of DNA mismatch repair in yeast. Science 264:1091-1093.

29. Strand, M., Prolla, T. A., Liskay, R. M., and Petes, T. D. Destabilization of tracts of simple repetitive DNA in yeast by mutations affecting DNA mismatch repair. 1993. Nature 365:274-276.

30. Studier, F. W. Use of bacteriophage T7 lysozyme to improve an inducible T7 expression system. 1991. J. Mol. Biol. 219(1):37-44.

31. Su, S. S., Lahue, R. S., Au, K. G., and Modrich, P. 1988. Mispair specificity of methyl directed DNA mismatch corrections in vitro. J. Biol. Chem. 263:6829-6835.

32. Nicolaides N C, Littnan S J, Modrich P, Kinzler K W, Vogelstein B 1998. A naturally occurring hPMS2 mutation can confer a dominant negative mutator phenotype. Mol Cell Biol 18:1635-1641.

33. Aronshtam A, Marinus M G. 1996. Dominant negative mutator mutations in the mutL gene of Escherichia coli. Nucleic Acids Res 24:2498-2504.

34. Wu T H, Marinus M G. 1994. Dominant negative mutator mutations in the mutS gene of Escherichia coli. J Bacteriol 176:5393-400.

35. Brosh R M Jr, Matson S W. 1995. Mutations in motif II of Escherichia coli DNA helicase II render the enzyme nonfunctional in both mismatch repair and excision repair with differential effects on the unwinding reaction. J Bacteriol 177:5612-5621.

36. Studamire B, Quach T, Alani, E. 1998. Saccharomyces cerevisiae Msh2p and Msh6p ATPase activities are both required during mismatch repair. Mol Cell Biol 18:7590-7601.

37. Alani E, Sokolsky T, Studamire B, Miret J J, Lahue RS. 1997. Genetic and biochemical analysis of Msh2p-Msh6p: role of ATP hydrolysis and Msh2p-Msh6p subunit interactions in mismatch base pair recognition. Mol Cell Biol 17:2436-2447.

38. Lipkin S M, Wang V, Jacoby R, Banerjee-Basu S, Baxevanis A D, Lynch H T, Elliott R M, and Collins F S. 2000. MLH3: a DNA mismatch repair gene associated with mammalian microsatellite instability. Nat Genet 24:27-35.

39. Lee C C, Lin H K, Lin J K. 1994. A reverse mutagenicity assay for alkylating agents based on a point mutation in the beta-lactamase gene at the active site serine codon. Mutagenesis 9:401-405.

40. Vidal A, Abril N, Pueyo C. 1995. DNA repair by Ogt alkyltransferase influences EMS mutational specificity. Carcinogenesis 16:817-821.

41. Fu, K. P., Grace, M. E., Hsiao, C. L., and Hung, P. P. 1988. Elimination of antibiotic-resistant plasmids by quinolone antibiotics. Chem. Abstracts 34(5):415-418.

42. BiWang, H., ZhiPeng, H., and Xiong, G. 1999. Transformation of Escherichia coli and Bacillus thuringiensis and their plasmid curing by electroporation. J. of Fujian Agricultural University 28(1):43-46.

43. Brosius, J. 1988. Expression vectors employing lambda-, trp-, lac-, and lpp-derived promoters. Biotechnology 10:205-225.

44. Alekshun, M. N. and Levy, S. B. 1999. Characterization of MarR Superrepressor Mutants. J. Bacteriol. 181:3303-3306.

What is claimed is:

1. A method for generating antibiotic resistant bacteria comprising the steps of:
blocking mismatch repair in a bacterium whereby said bacterium becomes hypermutable;
contacting said bacterium with at least one antibiotic;
selecting said a bacterium that is resistant to said antibiotic; and
culturing said bacterium;
thereby generating antibiotic resistant bacteria.

2. The method of claim 1 wherein said mismatch repair is blocked by introducing a dominant negative allele of a mismatch repair gene into said bacterium.

3. The method of claim 2 wherein said dominant negative allele of a mismatch repair gene is a PMS2-134 gene.

4. The method of claim 1 wherein said mismatch repair is blocked by introducing an antisense nucleic acid molecule into said bacterium wherein said antisense nucleic acid molecule specifically binds to a mismatch repair gene and inhibits mismatch repair in said bacterium.

5. The method of claim 1 wherein said mismatch repair is blocked by exposing said bacterium to a compound that inhibits mismatch repair.

6. The method of claim 5 wherein said compound is an anthracene derivative having the formula:
wherein R₁-R₁₀ are independently hydrogen, hydroxyl, amino, alkyl, substituted alkyl, alkenyl, substituted alkenyl, alkynyl, substituted alkynyl, O-alkyl, S-alkyl, N-alkyl, O-alkenyl, S-alkenyl, N-alkenyl, O-alkynyl, S-alkynyl, N-alkynyl, aryl, substituted aryl, aryloxy, substituted aryloxy, heteroaryl, substituted heteroaryl, aralkyloxy, arylalkyl, alkylaryl, alkylaryloxy, arylsulfonyl, alkylsulfonyl, alkoxycarbonyl, aryloxycarbonyl, guanidino, carboxy, an alcohol, an amino acid, sulfonate, alkyl sulfonate, CN, NO₂, an aldehyde group, an ester, an ether, a crown ether, a ketone, an organosulfur compound, an organometallic group, a carboxylic acid, an organosilicon or a carbohydrate that optionally contains one or more alkylated hydroxyl groups;
wherein said heteroalkyl, heteroaryl, and substituted heteroaryl contain at least one heteroatom that is oxygen, sulfur, a metal atom, phosphorus, silicon or nitrogen;
wherein said substituents of said substituted alkyl, substituted alkenyl, substituted alkynyl, substituted aryl, and substituted heteroaryl are halogen, CN, NO₂, lower alkyl, aryl, heteroaryl, aralkyl, aralkyloxy, guanidino, alkoxycarbonyl, alkoxy, hydroxy, carboxy and amino; and
wherein said amino groups optionally substituted with an acyl group, or 1 to 3 aryl or lower alkyl groups.

7. The method of claim 6 wherein said compound is selected from the group consisting of 1,2-dimethylanthracene, 9,10-dimethyl anthracene, 7,8-dimethylanthracene, 9,10-diphenylanthracene, 9,10-dihydroxymethylanthracene, 9-hydroxymethyl-10-methylanthracene, dimethylanthracene-1,2-diol, 9-hydroxymethyl-10-methylanthracene-1,2-diol, 9-hydroxymethyl-10-methylanthracene-3,4-diol, 9,10-di-m-tolyanthracene.

8. The method of claim 6, further comprising exposing said bacterium to a chemical mutagen.

9. The method of claim 8 wherein said chemical mutagen is selected from the group consisting of methane sulfonate, dimethyl sulfonate, O-6-methyl benzadine, ethylnitrosourea, ethidium bromide, ethyl methanesulfonate, N-methyl-N′-nitro-N-nitrosoguanidine, methylnitrosourea, Tamoxifen, and 8-hydroxyguanine.

10. The method of claim 5 wherein said compound is selected from the group consisting of an ATP analog, a nuclease inhibitor, and a DNA polymerase inhibitor.

11. The method of claim 10 wherein said ATP analog is selected from the group consisting of AMP-PNP and ATP[gamma]S.

12. The method of claim 10 wherein said nuclease inhibitor is selected from the group consisting of N-ethylmaleimide, heterodimeric adenine-chain-acridine compounds, exonulcease III inhibitors and heliquinomycin.

13. The method of claim 10 wherein said DNA polymerase inhibitor is selected from the group consisting of actinomycin D analogs, aphidicolin, 1-(2′-Deoxy-2′-fluoro-beta-L-arabinofuranosyl)-5-methyluracil, and 2′,3′-dideoxyribonucleoside 5′-triphosphates.

14. The method of claim 1 wherein said antibiotic is a quinilone.

15. The method of claim 1 wherein said antibiotic is an aminoglycoside.

16. The method of claim 1 wherein said antibiotic is a magainin.

17. The method of claim 1 wherein said antibiotic is a defensin.

18. The method of claim 1 wherein said antibiotic is a tetracycline.

19. The method of claim 1 wherein said antibiotic is a beta-lactam.

20. The method of claim 1 wherein said antibiotic is a macrolide.

21. The method of claim 1 wherein said antibiotic is a lincosamide.

22. The method of claim 1 wherein said antibiotic is a sulfonamide.

23. The method of claim 1 wherein said antibiotic is a chloramphenicol.

24. The method of claim 1 wherein said antibiotic is a nitrofurantoin.

25. The method of claim 1 wherein said antibiotic is an isoniazid.

26. The method of claim 1 wherein the step of determining whether said bacterium is resistant to said antibiotic comprises analyzing said bacterium for multiantiboitic resistance.

27. The method of claim 1 further comprising making antibiotic resistant bacteria genetically stable.

28. The method of claim 5 further comprising making antibiotic resistant bacteria genetically stable.

29. The method of claim 28 wherein said antibiotic resistant bacteria are made genetically stable by removing the MMR inhibitory molecule.

30. A method for identifying a mutant gene conferring antibiotic resistance comprising comparing the genome of antibiotic resistant bacterium made by the method of claim 1 to the genome of a wild-type strain of said bacterium.

31. The method of claim 30 wherein the genome of said antibiotic resistant bacterium and the genome of said wild-type strain of said bacterium are compared by sequence analysis of the entire genomes.

32. The method of claim 30 wherein the genome of said antibiotic resistant bacterium and the genome of said wild-type strain of said bacterium are compared by microarray analysis.

33. The method of claim 30 wherein the genome of said antibiotic resistant bacterium and the genome of said wild-type strain of said bacterium are compared by:
introducing gene fragments from said antibiotic resistant bacterium into the wild-type bacterium, thereby producing mutant bacteria;
selecting a mutant bacterium with antibiotic resistance; and
sequencing said gene fragment from said mutant bacterium with antibiotic resistance, thereby identifying the antibiotic resistance gene.

34. The method of claim 30 wherein the genome of said antibiotic resistant bacterium and the genome of said wild-type bacterium are compared by:
introducing gene fragments from said wild-type strain of said bacterium into the antibiotic resistant strain of said bacterium;
selecting a mutant bacterium with antibiotic resistance; and
sequencing said gene fragment from said mutant bacterium, thereby identifying the antibiotic resistance gene.

35. A method of producing an antibiotic resistant bacterium comprising the steps of:
culturing bacteria with a natural defect in mismatch repair;
contacting said bacteria with at least one antibiotic;
selecting a bacterium among said bacteria resistant to said antibiotic; and
culturing said bacterium;
thereby generating antibiotic resistant bacteria.

36. A method of generating antibiotic resistant bacteria comprising the steps of:
overexpressing a mismatch repair gene in a bacterium whereby said bacterium becomes hypermutable;
contacting said bacterium with at least one antibiotic;
determining whether said bacterium is resistant to said antibiotic; and
culturing said bacterium;
thereby generating antibiotic resistant bacteria.

37. The method of claim 36 further comprising making said antibiotic resistant bacteria genetically stable.

38. An antibiotic resistant bacterium produced by the method of claim 1.

39. An antibiotic resistant bacterium produced by the method of claim 35.

40. An antibiotic resistant bacterium produced by the method of claim 36.

41. An antibiotic resistant bacterium produced by the method of claim 37.
